(19) United States
(12) Patent Application Publication
(10) Pub. No.: US 2016/0262727 A1
(43) Pub. Date: Sep. 15, 2016

(54) ACOUSTIC DETECTION OF ACTIVATED PHASE-CHANGE CONTRAST AGENT

(71) Applicants: The University of North Carolina at Chapel Hill, Chapel Hill, NC (US); The Arizona Board of Regents on behalf of The University of Arizona, Tucson, AZ (US)

(72) Inventors: Paul Alexander Dayton, Carrboro, NC (US); Paul Steven Sheeran, Durham, NC (US); Brooks Daniel Lindsey, Durham, NC (US); Terry Onichi Matsunaga, Tucson, AZ (US); Juan David Rojas, Durham, NC (US)

(21) Appl. No.: 15/035,211

(22) PCT Filed: Nov. 10, 2014
(86) PCT No.: PCT/US2014/064889

§ 371 Date: May 6, 2016

Related U.S. Application Data
(60) Provisional application No. 61/902,150, filed on Nov. 8, 2013.

(51) Int. Cl. A61B 8/08; A61K 49/22; A61B 8/00

(52) U.S. Cl. CPC A61B 8/481; A61B 8/54; A61B 8/52; A61B 8/4477; A61B 8/461; A61K 49/223

(57) ABSTRACT

Methods, systems, and computer readable media for acoustic detection of activated phase-change contrast agents are disclosed. According to one aspect, a method for acoustic detection of activated ultrasound phase-change contrast agents includes initiating a phase transition in a phase change contrast agent causing a change of the diameter of the activated phase change contrast agent, and detecting an acoustic signature of the change of diameter of the activated phase change contrast agent.

### PRIORITY CLAIM

This application claims the priority benefit of U.S. Provisional Patent Application Ser. No. 61/902,150, filed Nov. 8, 2013, which is incorporated by reference herein in its entirety.

### GOVERNMENT INTEREST

This invention was made with government support under Grant No. DMR-1122483 awarded by the National Science Foundation and Grant No. EB011704 awarded by the National Institutes of Health. The U.S. Government has certain rights in the invention.

### TECHNICAL FIELD

The subject matter described herein relates to methods and systems for diagnostic and therapeutic applications of ultrasound. More particularly, the subject matter described herein relates to methods, systems, and computer readable media for acoustic detection of activated phase-change contrast agents.

### BACKGROUND

Phase-change contrast agents (PCCAs) have been gaining increasing attention in recent years as agents for diagnostic and therapeutic applications of ultrasound. PCCAs provide a dynamic platform to approach problems in medical ultrasound (US). Upon US-mediated activation, the liquid core vaporizes and expands to produce a gas bubble ideal for US imaging and therapy. Once activated, PCCAs can provide enhanced contrast for ultrasonic imaging, making them suitable for image-guided diagnostics and therapeutics. It would be useful to be able to determine exactly where and when the PCCAs change phase as well as how many of the PCCAs changed phase. There is currently no technique that can provide that information, however. Accordingly, there exists a need for techniques for detection of activated ultrasound phase-change contrast agents and the activation of same.

### SUMMARY

The subject matter described herein includes the acoustic detection of unique ‘acoustic signatures’ emitted by activated phase-change contrast agents as they convert from the liquid to gaseous state. Once activated by a primary source (such as ultrasound, x-ray, or laser), the vaporization event can be monitored passively by ultrasound and the signals differentiated from surrounding tissue and other ultrasound contrast agents. Detection of these signals allows spatial localization and quantification of activation, and may be useful for many diagnostic and therapeutic applications of ultrasound as well as other modalities, such as photoacoustic tomography.

According to one aspect, the subject matter described herein includes a method for acoustic detection of activated phase-change contrast agents. The method includes initiating phase transition in a phase change contrast agent causing a change of the diameter of the activated phase change contrast agent, and detecting an acoustic signature of the change of diameter of the activated phase change contrast agent.

According to another aspect, the subject matter described herein includes a system for acoustic detection of activated phase-change contrast agents and the activation of same. The system includes an energy source for providing activation energy that activates phase change contrast agents, causing a change of the diameter of the activated phase change contrast agents, and a detector for detecting an acoustic signature of the change of diameter of the activated phase change contrast agents.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

Preferred embodiments of the subject matter described herein will now be explained with reference to the accompanying drawings, wherein like reference numerals represent like parts, of which:

FIG. 1A is a block diagram illustrating an exemplary system for acoustic detection of activated phase-change contrast agents according to an embodiment of the subject matter described herein;

FIG. 1B is a flow chart illustrating an exemplary process for acoustic detection of activated phase-change contrast agents according to an embodiment of the subject matter described herein;

FIGS. 1C and 1D are graphs showing vaporization and expansion properties of volatile perfluorocarbon droplets vaporized with a 2-cycle sinusoid at 8 MHz according to an embodiment of the subject matter described herein;

FIG. 2 is a graph showing the relationship between final bubble diameter and initial droplet diameter for droplets of DFB (N=60) and OFP (N=43) according to an embodiment of the subject matter described herein;

FIG. 3 is a graph showing the oscillation frequency curve of resulting bubbles activated according to an embodiment of the subject matter described herein;

FIGS. 4A-4D are graphs showing control recordings with no bubbles or droplets present;

FIGS. 5A-5F are graphs showing individual voltage traces received from microbubble samples of varying concentration and associated spectra according to an embodiment of the subject matter described herein;

FIGS. 6A-6D are graphs showing signals produced by individual droplets vaporizing in the receive transducer's focus manifest as exponentially decaying sinusoids with very narrowband frequency content according to an embodiment of the subject matter described herein;

FIG. 7 is a graph showing that, when scaled to correct for the receiver transducer's frequency-dependent sensitivity, the amplitude of single OFP droplet signals can be related to the frequency of oscillation according to an embodiment of the subject matter described herein;

FIGS. 8A and 8B are graphs showing percentage of 200 sequential pulses where droplet signals are detected as a function of concentration and peak negative pressure of the incident 8 MHz ultrasound pulse according to an embodiment of the subject matter described herein; and

FIGS. 9A and 9B are graphs showing strength of detected droplet signals relative to 99th percentile microbubble (50% dilution) signal magnitude after processing according to an embodiment of the subject matter described herein.

### DETAILED DESCRIPTION

Phase-change contrast agents (PCCAs) provide a dynamic platform to approach problems in medical ultrasound (US). Upon US-mediated activation, the liquid core vaporizes and expands to produce a gas bubble ideal for US imaging and therapy. In this study, we demonstrate through high-speed video microscopy and US interrogation that PCCAs composed of highly volatile perfluorocarbons (PFCs) exhibit unique acoustic behavior that can be detected and differentiated from standard microbubble contrast agents. Experimental results show that when activated with short pulses PCCAs will over-expand and undergo unforced radial oscillation while settling to a final bubble diameter. The size-dependent oscillation phenomenon generates a unique acoustic signal that can be passively detected in both time and frequency domain using confocal piston transducers with an ‘activate high’ (8 MHz, 2 cycles), ‘listen low’ (1 MHz) scheme. Results show that the magnitude of the acoustic ‘signature’ increases as PFC boiling point decreases. By using a band-limited spectral processing technique, the droplet signals can be isolated from controls and used to build experimental relationships between concentration and vaporization pressure. The techniques shown here may be useful for physical studies as well as development of droplet-specific imaging techniques.

In an alternate embodiment, the activation frequency for the phase change contrast agent may be any frequency or frequencies selected from the range of 20 kHz to 100 MHz.

In accordance with the subject matter disclosed herein, methods, systems, and computer readable media for acoustic detection of activated phase-change contrast agents and the activation of same are provided.

The subject matter described herein includes findings that the phase-conversion process itself generates new acoustic waves as a result of particle over-expansion and oscillation. These new acoustic signatures can be detected and used to confirm the presence of particle phase-conversion in both time and spatially. Furthermore, the frequency of signals emitted are dependent on initial particle size, and so the methods outlined provide a way to detect signals confirming particles of specific sizes have vaporized—answering a direct need in the literature. This detection technique could be used to monitor PCCA therapy in real-time and lead to new transducer detection schemes for commercial diagnostic ultrasound systems and possibly industrial applications.

In this study, we demonstrate through high-speed microscopy and US interrogation that PCCAs composed of highly volatile perfluorocarbons (PFCs) exhibit unique acoustic behavior that can be detected and differentiated from standard microbubble contrast agents. Experimental results show that when activated with short ultrasound pulses, PCCAs will over-expand and undergo unforced radial oscillation while settling to a final bubble diameter. The size-dependent oscillation phenomenon generates a unique acoustic signal that can be passively detected in both time and frequency domain using confocal piston transducers with an ‘activate high’ (8 MHz, 2 cycles), ‘listen low’ (1 MHz) scheme. Results show that the magnitude of the acoustic ‘signature’ increases with PFC volatility. By using a band-limited spectral processing technique, the droplet signals can be isolated from controls and used to build experimental relationships between concentration and vaporization pressure. The techniques shown here may be useful for physical studies as well as development of droplet-specific imaging techniques.

Reference will now be made in detail to exemplary embodiments of the present invention, examples of which are illustrated in the accompanying drawings. Wherever possible, the same reference numbers will be used throughout the drawings to refer to the same or like parts.

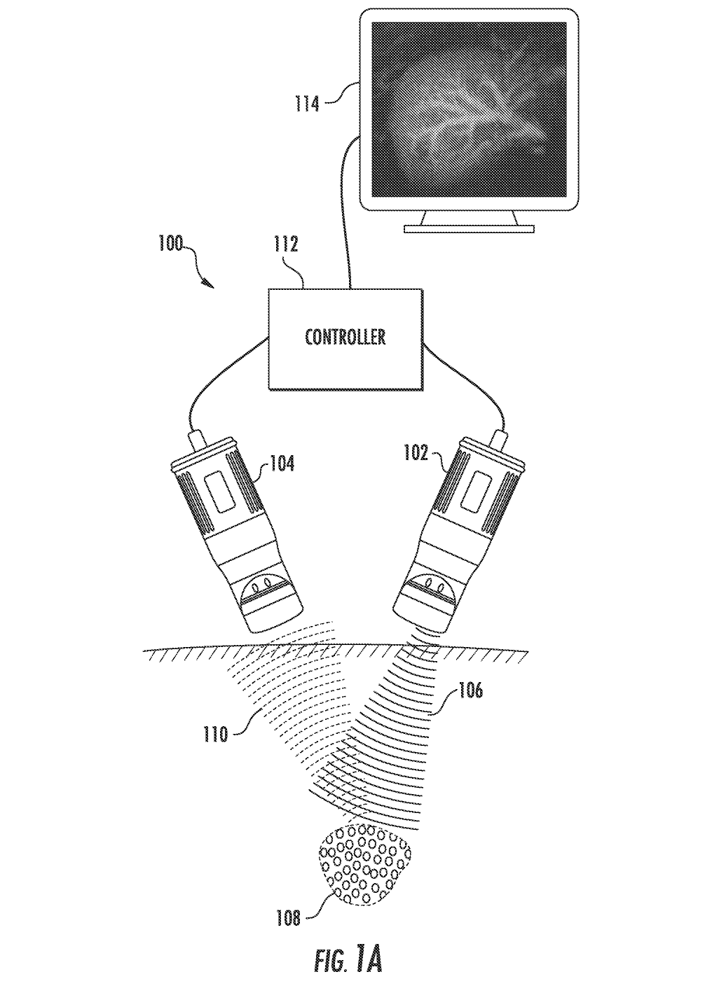

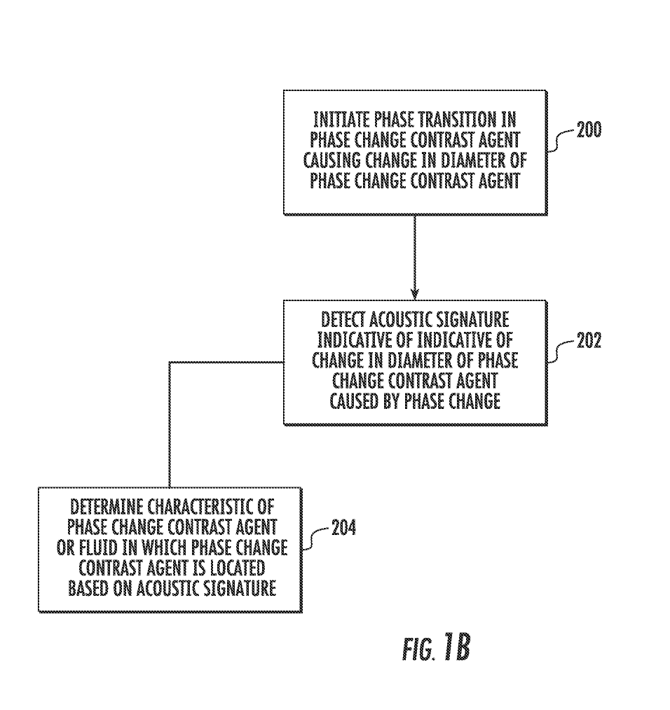

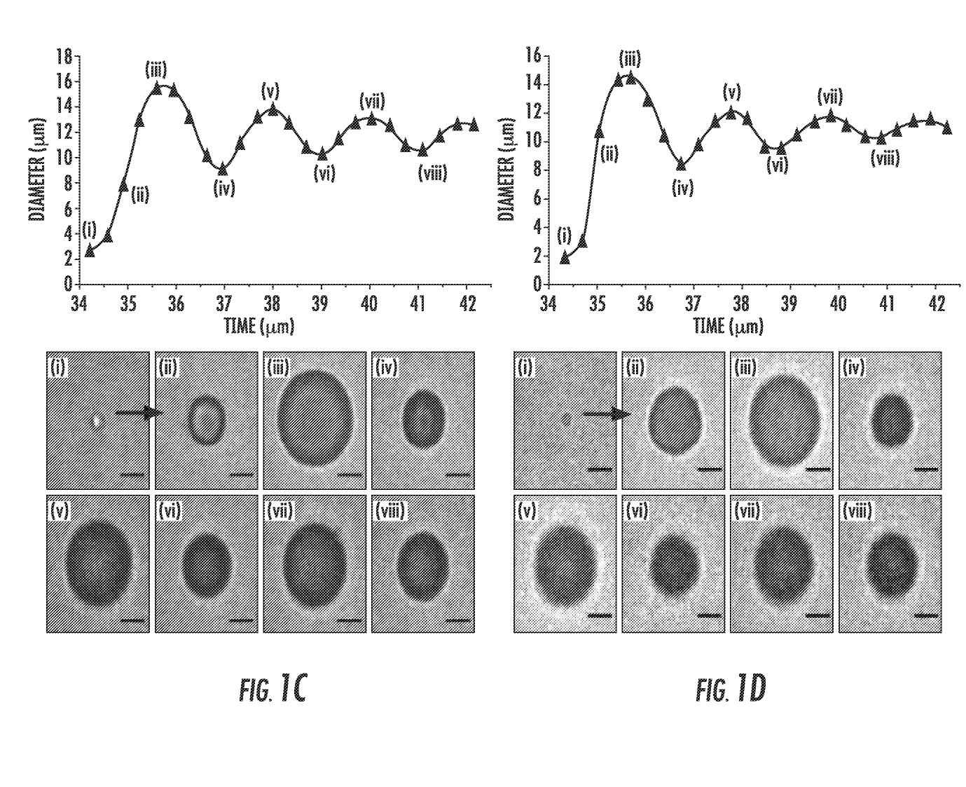

FIG. 1A is a block diagram illustrating an exemplary system for acoustic detection of activated phase-change contrast agents according to an embodiment of the subject matter described herein. In the embodiment illustrated in FIG. 1A, includes an energy source 102 for providing activation energy that activates phase change contrast agents, causing a change in the diameter of the activated phase change contrast agents, and a detector 104 for detecting an acoustic signature of the change of diameter of the activated phase change contrast agents. In one embodiment of the subject matter described herein, detector 104 may be configured to detect acoustic energy in a frequency range of 20 kHz to 100 MHz.

Energy source 102 may provide activation energy in the form of light, heat, mechanical, or other type of energy. In one embodiment, energy source 102 may provide ultrasonic energy 106 in a range, such as from 7 MHz to 8 MHz, that activates phase change nanodroplets 108 comprised of perfluorocarbons, hydrocarbons, hydrofluorocarbons, hydrohalocarbons, or other phase change contrast agents. Detector 104 may be an ultrasonic transducer able to detect the ultrasonic signatures 110 created by the activated phase change contrast agent. In one embodiment, acoustic signature 110 is a damped sinusoidal oscillation having a frequency in the 1 MHz-5 MHz range. The damped sinusoidal oscillation may have characteristic parameters, such as frequency, amplitude, or phase parameters that are usable as the signature of the phase change contrast agent. In some embodiments, detector 104 may also be the energy source, such as an ultrasonic transducer that can emit ultrasound in the activation frequencies and detect ultrasound in the signature frequencies. In one embodiment, a controller 112 may process the information detected by detector 104 into a form useful for diagnostics and/or therapeutics. For example, the location, signal magnitude, approximate number, and/or size of the activated phase change contrast agents may be presented to a user via a display 114. In another example, controller 112 may measure the change in acoustic signature over time to determine velocity and/or direction of a fluid in which the phase change contrast agent is located.

In one embodiment, if ultrasound energy is used to provide the activation energy source 102 and detector 104 may be the same or different ultrasound transducers. If different transducers are used, the transducers may have −6 dB or −12 dB bandwidths that do not overlap to allow the acoustic signatures of the activated phase change contrast agent to be detected with reduced noise from the activation energy.

Results achieved include optical characterization of particle activation via ultra-high-speed microscopy, characterization of particle resonance and size-dependence, as well as acoustic detection of signals from activated particles and comparison to controls.

FIG. 1B is a flow chart illustrating an exemplary process for detection of activated phase-change contrast agents according to an embodiment of the subject matter described herein. Referring to FIG. 1B, in step 200, a phase transition in a phase-change contrast agent is initiated causing the change of diameter of the phase-change contrast agent. For example, an ultrasound transducer may be used to activate nanodroplets 108 that have been injected into a patient to an area of the patient's anatomy. As stated above, the nanodroplets may be perfluorocarbons and the ultrasonic energy activation frequency may be in a range between 7 and 8 MHz.

In step 202, an acoustic signature indicative of the change in diameter of the phase change contrast agent caused by the phase transition is detected. For example, an ultrasound transducer may be used to detect the acoustic signature of nanodroplets 108 as they oscillate and change phase in response to the applied ultrasonic energy. The following description describes the use of high-speed video microscopy and ultrasound interrogation to detect acoustic signatures of PCCAs.

In step 204, a characteristic of the phase change contrast agent or the fluid in which the phase change contrast agent is located may be determined based on the acoustic signature. Step 204 may be performed by controller 112 illustrated in FIG. 1A. The characteristic may be the location of the phase change contrast agent, the signal magnitude, the size of the phase change contrast agent, the velocity of the fluid in which the phase change contrast agent is located, the direction of flow of the fluid in which the phase change contrast agent is located, or any of the other examples of characteristics described herein.

### 1. INTRODUCTION

The field of microbubble-based contrast agents for ultrasound has produced a host of applications ranging from diagnostic assessment of heart function to drug delivery and molecular imaging schemes that can be monitored in real-time (Gramiak and Shah 1968, Stride and Coussios 2010, Martin and Dayton 2013). Due to the underlying physics of oscillating microbubbles, bubble-specific detection techniques have been produced that exploit regimes of non-linear oscillation and/or nonlinear relationship to ultrasound driving pressure such that they can be distinguished from the ultrasound signals returned from tissues (Whittingham 2005). Use of these techniques has allowed real-time separation of contrast and tissue signals, enabling new modes of qualitative and quantitative imaging, many of which have been implemented on commercial clinical and pre-clinical ultrasound platforms (ten Kate et al 2010, Streeter et al 2010, Needles et al 2010, Gessner and Dayton 2010). Where microbubbles are used for therapeutic applications involving ultrasound-mediated ablation of tissue, the destruction of microbubbles creates highly nonlinear signals that can be detected over tissue signals as a general increase in the broadband spectral content. Quantifying this relative to the harmonics generated from the surrounding tissues allows real-time monitoring of cavitation during the ablation procedure and has produced some unique methods of imaging cavitation passively (Coussios and Roy 2008, Salgaonkar et al 2009, Choi and Coussios 2012, Choi et al 2007).

Within the past 15-20 years, researchers have begun to investigate alternative ultrasound contrast agents in order to overcome some of the fundamental limitations of microbubbles (such as limited size range and short in vivo half-life). Many of these studies have centered on the idea of a phase-change contrast agent (or PCCA) that converts from the liquid state to the gas state upon some external stimulus (Kripfgans et al 2000, Miller et al 2000, Sheeran and Dayton 2012, Rapoport 2012). By harnessing the spatial and temporal aspects of this transition, researchers can take advantage of the change in particle size, density, and compressibility for specific applications, such as temporary vessel occlusion, drug delivery, and enhancement of focused acoustic ablation (Kripfgans et al 2000, Rapoport et al 2009, Zhang and Porter 2010, Zhang et al 2011, Sheeran and Dayton 2012, Zhang et al 2010). Nearly all formulations of PCCAs to date have used perfluorocarbons (PFCs) with boiling points near body temperature as the liquid core component due to the fact that PFCs have relatively high molecular weight, low solubility, and low toxicity at the injected volumes (Mattrey 1994, Sheeran and Dayton 2012). Using these PFCs, particles generate relatively low ultrasound contrast while in the liquid state, as the density and compressibility are similar to surrounding tissue and blood. However, once vaporized, the gas bubble produced is an efficient scatterer of acoustic energy that results in ultrasound imaging contrast (Kripfgans et al 2000). Depending on the choice of perfluorocarbon, encapsulation, and particle size, vaporized droplets may either form stable microbubbles (Kripfgans et al 2000, Reznik et al 2011, Sheeran et al 2013), or may recondense to the liquid state after the vaporization pulse ends (Rapoport et al 2011, Reznik et al 2013, Asami and Kawabata 2012)—both of which have utility for specific applications.

Although real-time acoustic detection and isolation of microbubble-specific signals exists, there are currently few proposed methods to detect acoustic signals unique to PCCAs (that is, distinct from the surrounding tissue as well as distinct from standard microbubbles). Asami et al. have suggested that the impulse wavefront from droplets vaporizing adjacent to a rigid boundary could be detected and used to characterize tissue properties (Asami et al 2010). More recently, Reznik et al. have suggested that as bubbles produced from vaporized dodecafluoropentane (DDFP, boiling point 29° C.) droplets evolve over the course of several hundred milliseconds, the change in scattered fundamental and harmonic power over time could be used to differentiate growing bubbles (produced by recently vaporized droplets) from nearby stable bubbles (Reznik et al 2011). Development of a technique to detect unique signals produced by the droplet vaporization event would enable real-time imaging of droplet vaporization for diagnostic purposes as well as real-time monitoring of progress in therapeutic applications involving phase-change agents.

Some of the possibilities for acoustic detection of vaporized droplets may be learned from the underlying physical theory of expansion. A number of studies have modeled the physics of expansion after vaporization through theory and simulations in order to explore various aspects of parameter dependencies and in vivo implementation (Ye and Bull 2004, 2006, Qamar et al 2010, Eshpuniyani et al 2008, Calderon et al 2010, Shpak et al 2013b, 2013a, Pitt et al 2013, Qamar et al 2012). For those investigating droplet vaporization that produces stable bubbles, the models have generally predicted a monotonic expansion that can be modulated by the influence of the vaporization pulse and inward gas diffusion, which matches experimental data for DDFP microdroplets well (Shpak et al 2013b, 2013a, Wong et al 2011). For these types of droplets, the pressure wavefront generated by the initially high expansion velocities may be able to be detected as a sign of droplet vaporization—similar to the method proposed by Asami et al. (Asami et al 2010). Alternative models proposed by Qamar et al. (Qamar et al 2010, 2012) have suggested that, depending on droplet size and other physical properties, droplets may over-expand as a result of momentum of expansion and then settle to a final resting diameter in an oscillatory manner, although the experimental results with DDFP microdroplets did not exhibit this behavior. If this type of behavior could be achieved experimentally, it might be possible to detect these oscillations as an acoustic signature of droplet vaporization.

Recent studies by our group have produced a method to develop PCCAs from highly volatile perfluorocarbons that are inherently more sensitive to ultrasound energy than those formed similarly from DDFP or perfluorohexane (PFH, boiling point 56° C.) (Sheeran et al 2011b, 2011a, 2012). In this context, PFC volatility refers to the degree to which the boiling point of the PFC used is less than room temperature (22° C.). When emulsified to diameters in the nanoscale, the additional Laplace pressure exerted on the droplet core raises the boiling point enough to create droplets that remain metastable in the liquid state at body temperature, even when formed out of compounds such as decafluorobutane (DFB, boiling point −2° C.) and octafluoropropane (OFP, boiling point −36.7° C.). Although an inherent tradeoff exists between sensitivity to ultrasound and thermal stability (and, therefore, circulation half-life), creating PCCAs from perfluorocarbon mixtures allows ‘fine-tuning’ the balance of these competing factors for specific applications (Sheeran et al 2012).

In this study, we first demonstrate through ultra-high-speed brightfield video microscopy that when vaporized by a brief US pulse, volatile PCCAs at micron and sub-micron sizes exhibit expansion phenomena after the pulse has ended including over-expansion and oscillatory settling to a stable bubble size. We next show that this radial expansion/oscillation generates unique droplet-specific pressure signals that can be acoustically detected and differentiated from microbubble-based contrast agents and tissue with respect to both temporal and spectral content. These findings may be useful for physical studies as well as development of droplet-specific imaging techniques for medical imaging and therapy.

### 2. METHODS

### 2.1. Phase-Change Contrast Agent Preparation

To generate lipid-coated microscale and sub-micron droplets from volatile perfluorocarbons, a previously described ‘microbubble condensation’ method was used (Sheeran et al 2011b, 2011a, 2012). This simple, high-yield technique involves first forming polydisperse microbubble emulsions with a 9:1 M dissolution of 1,2-distearoyl-sn-glycero-3-phosphocholine (DSPC) and 1,2-distearoyl-sn-glycero-3-phosphoethanolamine-N-methoxy(polyethylene-glycol)-2000 (DSPE-PEG2000) for a total lipid concentration of 1 mg/mL in an excipient solution of phosphate-buffered saline (PBS), propylene glycol, and glycerol (16:3:1). All lipids were purchased from Avanti Polar Lipids (Alabaster, Ala.). From this lipid solution, 1.5 mL was pipetted into a 2 mL glass vial (Wheaton Industries, Millville, N.J.), and the air headspace replaced with perfluorocarbon gas (Fluoromed, Round Rock, Tex.). Bubbles were formed by mechanical agitation (Vialmix, Bristol-Myers-Squibb, New York, N.Y.), resulting in a high-yield, polydisperse microbubble distribution. The gas headspace was composed of either pure decafluorobutane (DFB, boiling point −2° C.) or pure octafluoropropane (OFP, boiling point −36.7° C.). The microbubble emulsions were allowed time to cool to room temperature, and then were immersed in an isopropanol bath maintained between −7° C. and −10° C. and stirred gently by mechanical rotation for approximately 1 minute. A change in consistency and translucency was typically noted by this point for DFB emulsions, though OFP emulsions could not be condensed by temperature alone. Vials were then connected to an adjustable pressure source and the pressure in the vial headspace was increased incrementally with room air while the low temperature was maintained to ensure particles reverted to the liquid state as previously described (Sheeran et al 2012). The combination of propylene glycol, glycerol, and PBS in the excipient solution prevented the emulsion from freezing due to the reduced temperatures. Vials were then stored at 4° C. for no more than 2 hours prior to use. Droplets were sized with dynamic light scattering (Malvern Nano ZS, Malvern Instruments Ltd., Malvern, Worcestershire, U.K.) by placing approximately 1.5 mL of a 10% dilution of the droplet emulsion in a plastic sizing cuvette. Nanoemulsions yielded consistent size distributions with a peak near 164 nm and a mean near 192 nm for number-weighted measurement; a peak near 190 nm and a mean near 297 nm for intensity-weighted measurement (Sheeran et al 2013). Conveniently, the emulsions also contain a small number of droplets on the order of 800 nm or greater that can be resolved optically and studied.

### 2.2. High-Speed Video Experimental Setup

The experimental setup used here was similar to that described in earlier studies (Dayton et al 2002, Sheeran et al 2011 b, 2011 a, 2012, 2013). An acrylic-lined, continuously degassed water bath was mounted onto an inverted microscope (Olympus IX71, Center Valley, Pa.), and the water in the bath was passively heated to 37° C. in order to test the emulsions at physiological temperatures. Brightfield images were captured using a 100× (NA=1.0) water immersion objective interfaced with an ultra-high-speed framing camera (SIMD24, Specialised Imaging, Simi Valley, Calif.) capable of capturing 24 images at up to 200 million frames per second. A xenon strobe was interfaced with the microscope system to provide high intensity illumination. A calibrated needle hydrophone (HNA-0400, Onda Corp., Sunnyvale, Calif.) was used to place the focus of the piston transducer confocal with the optical focus. The piston transducer was driven by an arbitrary waveform generator signal (AFG 3101, Tektronix, Inc., Beaverton, Oreg.) and amplified 60 dB using an RF amplifier (A500, ENI, Rochester, N.Y.). Acoustic pulses were manually triggered and synchronized with the high-speed camera in order to simultaneously capture video. For ultra-high-speed imaging studies, droplets were vaporized with a 7.5 MHz nominal center frequency spherically focused transducer (2.2 cm diameter, 5 cm focus; V321, Panametrics, Inc., Waltham, Mass.) driven with an adjustable-amplitude 2-cycle sinusoid at 8 MHz (insonification time of approximately 250 ns). The camera frame spacing was set to record evenly spaced captures within the imaging period (30-40 ns exposure times, maximum 20 μs of continuous filming time allowed by flash duration). The brief, high-intensity acoustic pulses were typically in the non-linear regime, and so transducers were calibrated in the free-field at focus using a needle hydrophone (HNA-0400, Onda Corp., Sunnyvale, Calif.) with a magnitude-only hydrophone deconvolution method (Hurrell 2004).

Droplet suspensions diluted to 10% in PBS were injected into a nearly optically and acoustically transparent 200 μm inner diameter microcellulose tube (outer diameter of 216 μm; Spectrum Laboratories, Inc., Greensboro, N.C.) resting in the confocal acoustic/optical plane. The droplet emulsions were injected via an in-house manual volume injector and the forward flow stopped prior to the manually triggered vaporization pulse. The imaging frame rate was adjusted in order to capture the size-dependent droplet vaporization sequences in high detail, as smaller droplets finish vaporizing more rapidly than large droplets (Sheeran et al 2013). Between video captures, forward flow was restored in order to bring un-vaporized agents into the optical focus. Images were stored offline and analyzed in ImageJ (NIH, Bethesda, Md.). Although the setup produced images with 15.58 pixels/μm, the practical optical resolution of the system due to image noise at the low exposure times limited measurement of particles to those greater than approximately 1 μm in diameter. The area of the droplets/bubbles in each frame were measured by hand using elliptical ROls and converted to diameter to track the change in size due to vaporization. This provided up to 24 measurements (the total number of frames) per video, which were then interpolated and analyzed with custom scripts in Matlab (The Mathworks, Natick, Mass.) to extract information such as rate of radial expansion, maximum expansion, final diameter, and the fundamental frequency of oscillation based on Fourier analysis of the radial oscillation. All statistical analysis was performed using a student's t-test (two sided, unequal variance).

### 2.3. Acoustic Interrogation Experimental Setup

The experimental setup used to capture echoes emitted from vaporizing droplets was similar to that used for high-speed imaging of droplet vaporization. A microcellulose tube containing droplets was placed at the optical focus of the heated, degassed water bath described previously. In order to minimize reflections from the microcellulose interface, the tube was angled 66° relative to the face of the transducer, while keeping the tube length in a plane perpendicular to the microscope objective. Instead of a single piston transducer aligned at the optical focus, a confocal dual-transducer approach was used in order to vaporize the droplets at the optical focus using a brief, higher frequency pulse, and passively listen to echoes emitted with a lower frequency transducer having a non-overlapping bandwidth with the activation transducer. This is analogous to, but opposite of the ‘pulse low, listen high’ scheme used in other studies to isolate bubble signals from tissue (Bouakaz et al 2002, Kruse and Ferrara 2005, Gessner et al 2010). Here, the ‘pulse high’ transducer used to activate droplets was a spherically focused piston transducer with a 7.5 MHz nominal center frequency (1.58 cm diameter, 5.08 cm focus; V320, Panametrics, Inc., Waltham, Mass.) driven with a 2-cycle sinusoid at 8 MHz (PRF of 5 Hz). In order to ‘listen low’, a spherically focused 1 MHz annular transducer (2.2 cm inner diameter, 4.44 cm outer diameter, 5.08 cm focus; V30047, Panametrics, Inc., Waltham, Mass.) was fitted around the ‘pulse high’ transducer and focused at the same spatial location (the optical focus containing the microcellulose tube) in order to passively receive echoes. As above, transducers were calibrated in the free-field at focus using a needle hydrophone (HNA-0400, Onda Corp., Sunnyvale, Calif.) to obtain both pressure output information as well as frequency response characteristics. The transmit-bandwidth of the activation transducer had a full-width half-maximum (FWHM) between 6.067 MHz and 10.933 MHz, while the FWHM of the receiving transducer in transmit-receive mode was between 700 kHz and 1.551 MHz. The output of the 1 MHz transducer was sent to a receiver amplifier (BR-640A; RITEC, Inc., Warwick, R.I.) set to pass signals above 100 kHz with a gain of 32 dB. The signals at the output of the receive amplifier were digitized at a 100 MHz sample rate (PDA14; Signatec, Ill.), captured in LabVIEW (National Instruments, Austin, Tex.), and stored for offline analysis in Matlab. Samples were injected at a constant flow rate driven by a syringe pump (PHD2000, Harvard Apparatus, Holliston, Mass.) at a rate of 10 μL/min in order to ensure that the volume exposed to the previous vaporization pulse translated a distance of at least 10 times the vaporization transducer's lateral pressure field FWHM prior to the next pulse. A range of ultrasound pressures (between 100 kPa and 2500 kPa peak negative pressure) were delivered from the activation transducer in order to assess the threshold-dependence of the droplet vaporization signals. Two hundred instances of the ‘pulse high, listen low’ scheme were captured at each pressure for each sample passing through the optical focus (at the pulse rate of 5 Hz). Droplet emulsions were diluted in PBS to final concentrations of 0.05%, 0.1%, 0.5%, 1%, 5%, 10%, and 50% in order to determine the influence of PCCA concentration on the returned acoustic signals. For comparison, similar data were captured for the cellulose tube when filled with only water, filled with only air, and filled with the same dilutions of microbubble contrast agents as used for the droplet emulsions. The microbubble emulsions used here consisted of the same lipid-encapsulated DFB microbubble precursors used to form DFB droplets, but without the additional step of condensation. Throughout these acoustic tests, the contents of the microcellulose tube were monitored in real-time by a high-speed camera (FastCam SA1.1, Photron USA, Inc., San Diego, Calif.) interfaced with a 60× water immersion objective. Radio-frequency (RF) signals captured from these tests were analyzed offline in Matlab for both time and frequency-domain characteristics in order to distinguish acoustic properties unique to droplet vaporization. For controls, each signal consisted of a total acquisition time of 40 μs with the travel time to the focus of the transducer occurring approximately 20 μs after the beginning of acquisition. For droplet samples, each signal consisted of a total acquisition time of 60 μs beginning at the same time point as the control samples. Prior to analysis, all signals were bandpass filtered (between 50 kHz and 5 MHz) with a 100-order FIR Butterworth filter in Matlab to remove broadband noise introduced by the experimental setup, as well as any signals far outside the bandwidth of the 1 MHz receiving transducer. No windowing was used on the data during frequency analysis.

### 3. RESULTS AND DISCUSSION

In the following results and discussion, results from ultra-high-speed optical imaging experiments were used to develop the acoustic interrogation approach. The acoustic interrogation results, in turn, were used to implement a simple droplet-specific algorithm capable of isolating droplet signals from control cases.

### 3.1. Ultra-High-Speed Video Experiments

Investigations with ultra-high-speed imaging showed that droplets of the formulations used were able to be vaporized with 2-cycle pulses at 8 MHz, which is consistent with earlier studies (Sheeran et al 2012, 2013). The pulses used to vaporize droplets had peak negative pressures on the order of 2.5 MPa for octafluoropropane droplets, and between 3.7 and 4.2 MPa for decafluorobutane droplets. It is worth noting that the pressures in this set of experiments were chosen because they were higher than necessary to induce vaporization rather than being indicative of any specific activation threshold for the emulsion. Because a short vaporization pulse was used that lasted less than 250 ns, and because droplets large enough to be resolved in the optical setup took longer than 1 μs to complete their initial expansion phase, the pulse primarily served to initiate the bubble nucleation, but did not influence bubble growth as a longer pulse would.

When activated with these short 8 MHz pulses, the transition from droplet to bubble was captured, and the diameter of the particle measured at each time point. The droplets showed unique vaporization properties not previously reported for droplets composed of less volatile compounds. During vaporization, particles showed characteristic over-expansion and subsequent oscillatory settling to a final bubble diameter smaller than the maximum size reached during the initial expansion phase (FIGS. 1C and 1D). This type of activity was observed for both OFP and DFB droplets ranging from the smallest size observable to the largest size present in the samples. The overexpansion and oscillation are expected to be a result of momentum of expansion leading to overshoot of the final diameter and oscillatory return to the final bubble size. This type of behavior has been previously predicted in simulations for vaporizing droplets under certain conditions (Qamar et al 2010).

FIGS. 1C and 1D illustrate vaporization and expansion properties of volatile perfluorocarbon droplets vaporized with a 2-cycle sinusoid at 8 MHz. As illustrated in FIG. 1C, a DFB droplet near 2.7 μm in diameter vaporizes and expands to a maximum near 15.5 μm in diameter within 2 μs, and eventually settles to a smaller resting diameter. As illustrated in FIG. 1D, an OFP droplet near 2 μm in diameter expands to a maximum near 14.6 μm in diameter and settles to a smaller resting diameter. In both cases, the droplet oscillations occurred over the course of 10 μs after vaporization. Scale bar represents 5 μm.

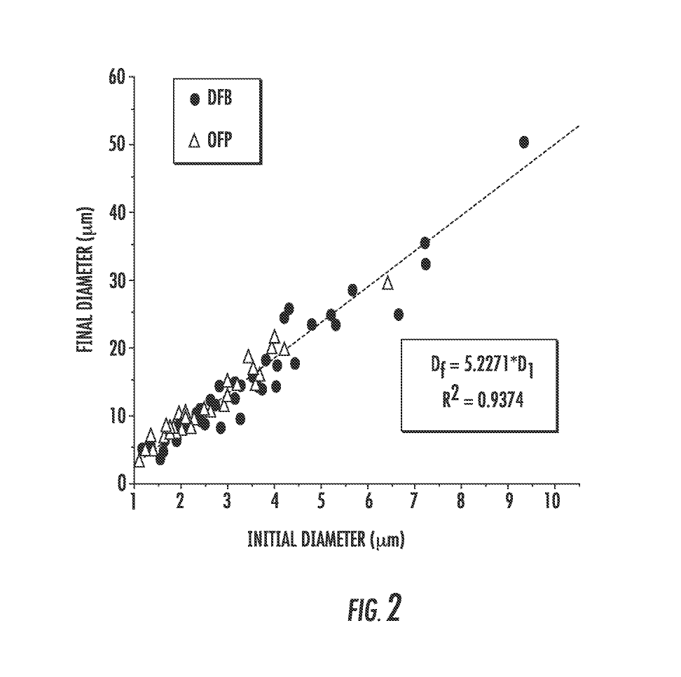

This type of expansion behavior was captured for a total of 180 OFP and DFB droplets and separated into sub-groups for analysis depending on whether certain aspects of the expansion were resolvable/measurable based on visual assessment. If the initial droplet size was well-resolved against background but the bubble expansion was not, the initial time points in the individual trace were omitted from measurement. Similarly, if the particle vaporization and initial expansion was well resolved, but during the course of expansion the bubble oscillation was perturbed by neighboring bubbles, measurements were only taken up to the point of perturbation and the remaining points omitted. Of the data produced, sub-groups were determined by assessing (1) whether the initial droplet size could be resolved with high confidence, (2) whether the initial expansion phase was well sampled, (3) whether the overshoot and oscillation behavior was captured and well-sampled, and (4) whether the final bubble size was captured or able to be predicted from oscillation behavior. During the initial expansion phase, DFB droplets over-expanded to 6.3±1.2 times the initial droplet diameter (N=76), while OFP droplets (N=52) over-expanded to 8.3±1.3 times the initial droplet diameter. The over-expansion of the OFP droplet group was statistically significant compared to the DFB droplet group (p<<0.01), and the expansion ratio did not appear to be dependent on initial diameter for either group in the range tested. The relationship between initial droplet size and final bubble size followed a strongly linear trend in the range tested (FIG. 2). The linear regression for DFB particles (N=60) showed a ratio of approximately 5.12 (r²=0.94, 95% confidence intervals of 4.94 and 5.30, RMSE=2.06) between final and initial diameter, while OFP particles (N=43) showed a ratio of approximately 5.53 (r²=0.93, 95% confidence intervals of 5.33 and 5.72, RMSE=1.38). The expansion of DFB particles was statistically significant compared to the expansion of OFP particles (p<<0.01). These experimental results match well with ideal gas law predictions for DFB and OFP expansion (Sheeran et al 2011b). For example, DFB droplets measuring 1 micrometer in diameter at 37° C. would theoretically expand a factor between 4.64 (assuming air/water interfacial surface tension of 71 mN/m) and 5.43 (negligible surface tension) times the original diameter, while identical droplets of OFP would expand a factor between 5.06 (air/water surface tension) and 5.88 (negligible surface tension) times the original diameter. During the initial expansion phase, peak radial expansion velocity in the imaging plane reached 9.8±2.7 m/s (N=84) for DFB and 13.9±3.9 m/s (N=52) for OFP. The OFP velocities were statistically significant from the DFB velocities (p<<0.01), and both groups appeared to be independent of initial droplet size in the range tested.

FIG. 2 illustrates a relationship between final bubble diameter and initial droplet diameter for droplets of DFB (N=60) and OFP (N=43). The strong linear trends showed a ratio of 5.12 between final and initial size for DFB (r²=0.94) and a ratio of 5.53 between final and initial size for OFP (r²=0.93). These experimental values matched well with ideal gas law predictions (see text) and appeared to be independent of initial diameter in the range tested.

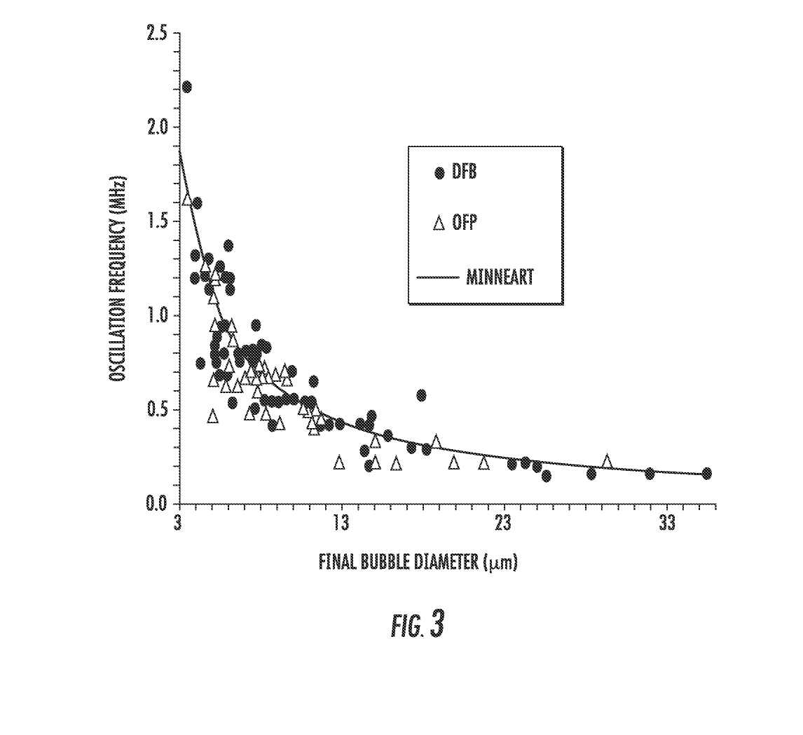

Results showed that as vaporized droplets over-expanded and oscillated in a decaying manner to a final resting bubble size, the frequency of oscillation was inversely dependent on final bubble size (and therefore initial droplet size). Large bubbles oscillated at low frequencies near 100 kHz compared to small bubbles that oscillated above 1 MHz. This size-frequency relationship is characteristic of a resonance phenomenon. In this case, the resonance was initiated by the overexpansion event in the initial vaporization phase (FIGS. 1C and 1D). By collecting these data over a wide range of particle sizes, a final bubble size vs. resonance frequency curve can be experimentally developed to predict the frequency of echoes emitted by a particle (FIG. 3). Although separate curves can be developed for the DFB and OFP droplet groups, the error in measured frequency imposed by the small number of frames limits any comparisons made between the groups. Combining the data and produces a fit of f(r)=2.679/r, where f is the measured frequency and r is the final bubble radius (r²=0.79, 95% confidence intervals of 2.578 and 2.779, RMSE 1.658×10⁵). FIG. 3 illustrates an oscillation frequency curve of resulting bubbles. As a function of final bubble radius, the particles oscillated at specific frequencies after the initial over-expansion phase. For the OFP (N=49) and DFB (N=81) particles tested that were optically resolvable, these were on the order of 100 kHz to 2.5 MHz, depending on bubble size. Curve-fitting the data produces a relationship of f(r)=2.679/r where f is the measured oscillation frequency and r is the final bubble radius (r²=0.79). Approximating the data with a Minnaert free bubble resonance model produces a slightly worse fit (r²=0.77).

Because the oscillation happened well after the vaporization pulse has passed, it was independent of the pulse, and represents natural resonance (rather than ‘driven’ or ‘forced’ oscillations). This is unique with respect to ultrasound contrast agents, in that the particle is ‘tipped’ and then emits an entirely original acoustic signal that does not overlap with the acoustic pulse temporally or spectrally, which is radiated and received by the ultrasound transducer. This is similar in some aspects to the broadband post-excitation signals produced by microbubbles in prior studies (King et al 2010, King and O'Brien 2011) that are associated with bubble collapse/inertial cavitation as a result of high intensity pulses. In contrast, the droplet-based signals shown here occur during stable bubble formation, and the primary mechanism that produces the relatively narrowband droplet-based signals is the overexpansion of the droplet during vaporization (independent of the vaporization pulse). The classic Minnaert equation for resonance of a free bubble (Minnaert 1933, Ainslie and Leighton 2011) is:

$${f}_{M} = \frac{1}{2 π r} \sqrt{\frac{3 κ {P}_{l}}{{ρ}_{l}}} \qquad ( 1 )$$

where f_{M} is the natural resonance frequency, r is the radius of the bubble, κ is the polytropic exponent, Pₗ is the ambient pressure, and ρₗ is the density of the surrounding liquid. Here, assuming a polytropic index of κ=1 (isothermal process), Pₗ=101.325 kPa (ambient pressure), and ρₗ=994 kg/cm³ (approximate density of PBS at 37° C.) produces a resonance curve that represents the experimental data nearly as well as the optimal experimental fit (r²=0.79, RMSE 1.658×10⁵), and can be used for simple approximations of the bubble size/resonance frequency. However, this ignores several aspects of the experimental system that deviate from the Minnaert model. An isothermal assumption for the polytropic index is not suitable for the size of microbubbles and the perfluorocarbon core of the bubbles. Most studies have suggested that adiabatic assumptions (κ=Cₚ/Cᵥ, the ratio of specific heat at constant pressure to specific heat at constant volume) model the behavior of perfluorocarbon microbubbles well (Doinikov and Bouakaz 2011), which would produce values of κ=1.052 for DFB and κ=1.063 for OFP at 37° C. (Linstrom and Mallard 2013). Using an approximate value of 1.06 for the combined data set, the Minnaert fit to the data is shifted slightly above the experimental data fit (FIG. 3, r²=0.77, RMSE=1.735×10⁵). The damping in the bubble oscillation accounts for the deviation between the experimental and ideal fits. For a classic damped harmonic oscillator undergoing un-forced oscillation, the decay of the system can be related to the measured (damped) oscillation frequency and ideal (Minnaert) frequency by:

$${f}_{damp} = \sqrt{{f}_{M}^{2} - {( \frac{β}{2 π} )}^{2}} \qquad ( 2 )$$

where β⁻¹ is the time constant of the decay in the exponentially decaying harmonic oscillation (Leighton 1994). The total damping constant of a freely oscillating bubble at resonance is the sum of radiation, thermal, and viscous damping, and can be related to β by:

$$δ = \frac{2 β}{{ω}_{damp}} = \frac{β}{π {f}_{damp}} \qquad ( 3 )$$

Substitution of Eqns. 1 and 3 into Eqn. 2 gives:

$${f}_{damp} = {f}_{M} \sqrt{\frac{1}{1 + {( \frac{δ}{2} )}^{2}}} \qquad ( 4 )$$

Fitting Eqn. 4 to the experimental data gives a total damping coefficient of δ=0.76 for the oscillations produced from phase-change droplets (r²=0.79, 95% confidence intervals of 0.53 and 0.98, RMSE=1.658×10⁵).

For the micron-sized particles characterized in these tests, the majority of particles exhibited oscillation frequencies between 100 kHz and 1 MHz, which is well below the bandwidth of the transducer delivering the activation pulse. Provided the new acoustic signals generated are strong enough to be detected, they could be picked up by an ultrasound transducer and used to localize the droplet activation event both temporally and spatially.

### 3.2. Acoustic Interrogation Results

To determine whether acoustic signals generated by vaporizing droplets could be detected, a ‘pulse high, listen low’ scheme was used that allowed for receiving low frequency acoustic signals not strongly related to the fundamental activation pulse. Based on the video data, any signals emitted from droplets oscillating on the order of 100 kHz to 1 MHz would appear as distinct spectral content relative to normal microbubble signals or tissue signals. To test this, the microcellulose tube setup previously described was injected with droplets at a constant flow rate and exposed to a series of 2-cycle pulses at 8 MHz. For controls, the same tube was filled with either water, air, microbubble dilutions. The water and air cases represented extremes of the microbubble dilutions—where there are so few bubbles present that the suspension acoustically appears as water, or when there are so many present that it appears as a solid air interface.

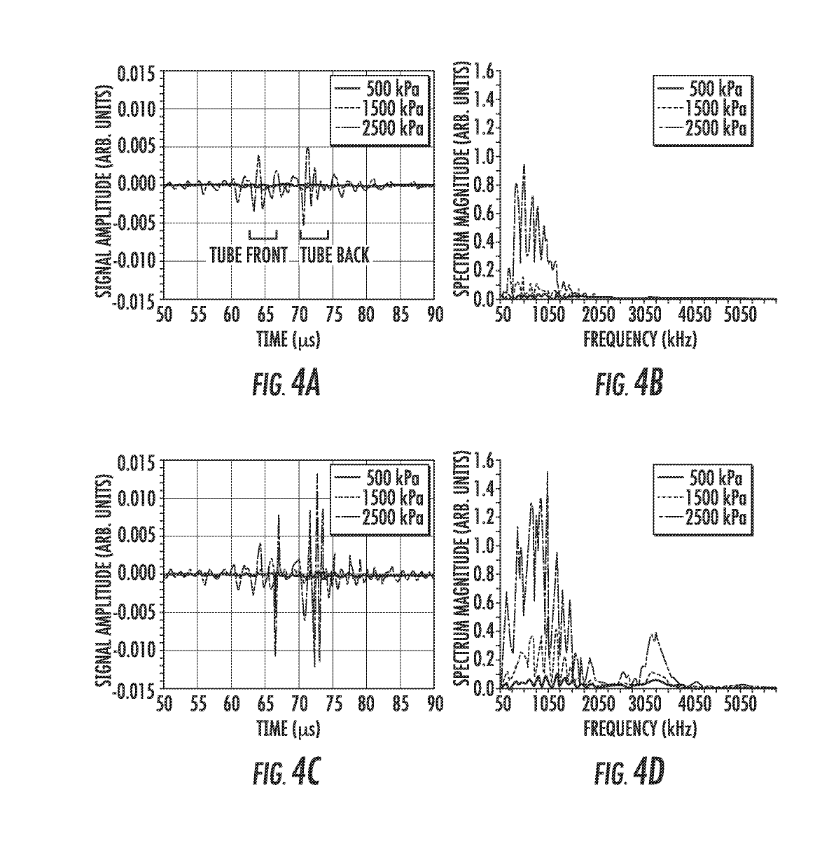

FIGS. 4A-4D illustrate—control recordings with no bubbles or droplets present. Echoes reflected from the microcellulose tube are apparent in the received signals on the 1 MHz transducer beginning near 60 μs from the pulse trigger, while a later echo is produced from the nearby microscope objective beginning near 70 μs. As illustrated in FIG. 4A, the water-microcellulose interface creates a small reflection when the tube is filled with water that manifests as noisy frequency content in the 50-2000 kHz range (FIG. 4B), whereas FIG. 4C illustrates that the water/microcellulose/air interface creates a very large reflection for a tube filled with air that manifests as noisy frequency content in the 50-2000 kHz range with an additional harmonic peak between 3-3.6 MHz (FIG. 4D). The final echo from the microcellulose tube in both cases returns near 70 μs, and the final echo from the objective artifact returns near 75 μs. Note: FIGS. 4A and 4C illustrate the mean of 200 captures of the water-only and air-only cases. FIGS. 4B and 4D show the Fast-Fourier Transform of the data presented in FIGS. 4A and 4C.

When the tube was filled with only water (FIG. 4A), the receiving transducer showed weak reflections of the activation pulse due to the interface of the water and capillary tube (a significant enough density difference to reflect some energy). Increasing the activation pressure only increased the amplitude of these pulses, but the frequency content remained noisy (FIG. 4B). Even though the activation pulse was well out of the frequency response of the 1 MHz transducer, the tube interface reflects back enough of the short burst of energy to ring the 1 MHz transducer near its own resonance frequency with a very minor harmonic component between 3 MHz and 3.5 MHz. This effect was amplified in the case of the ‘air-only’ tube, where the interface formed by the air produced a strong reflection off the tube (FIG. 4C). Similar to the case of water, the reflection off of the tube/air interface produced signals that cause the transducer to ring at 1 MHz with a small harmonic peak present between 3 MHz and 3.5 MHz (FIG. 4D). As the incident pressure increased, the magnitude of the frequency domain signal increased, but the fundamental frequency components remained relatively similar. In both the air and water cases, the only acoustic energy produced was from the activation pulse directly reflecting off of interfaces. In this experimental setup, an additional acoustic artifact was introduced by the nearby objective used to monitor particle flow, which manifested as an echo beginning a few microseconds after the end of the tube reflection (FIG. 4A,C). Once the final reflection off of this acoustic artifact returned (around 75 μs), no acoustic signals were detected for the remaining period.

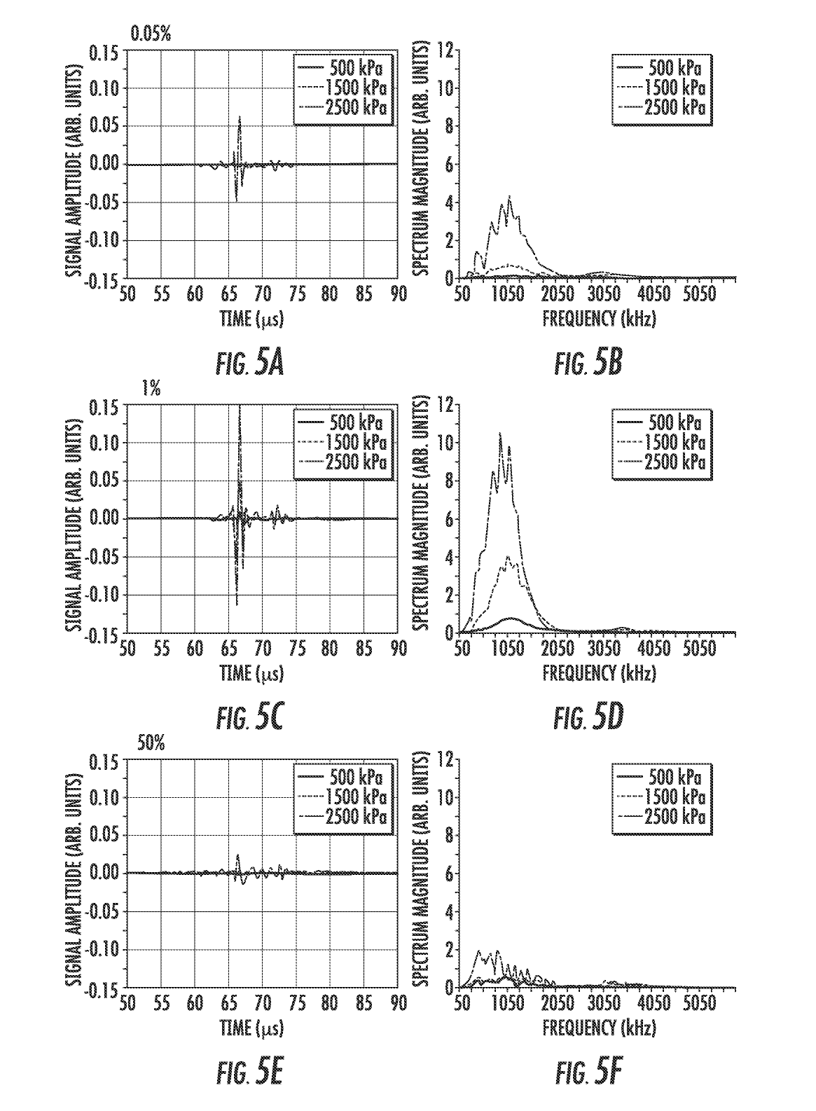

When the tube was filled with standard formulation microbubble contrast agents, the time and frequency domain signals were appreciably different from the air-only and water-only cases. At very low bubble concentrations, a sinusoidal trace was observed between 66 μs and 68 μs (corresponding with the temporal location of the tube reflection in FIGS. 4A-4D) that increased with the incident pressure (FIG. 5A). The temporal length of this signal corresponds well with the expected tube diameter when the receive transducer's impulse response is taken into account. Unlike the noisy spectra from the air-only and water-only cases, the bubble signals returned spectra that strongly resemble the receiving transducer's frequency response centered near 1 MHz (FIG. 5B,D,F). This is likely due to the fact that the short 8 MHz burst causes bubbles to oscillate (or become destroyed at higher pressures) and return a brief burst of energy back to the receive transducer that causes it to ring at its center frequency (similar to an impulse response). At the concentrations tested, the magnitude of the time domain signal increased with microbubble concentration up to 1% dilution (FIG. 5C), past which the signal magnitude decreased rapidly up to 50% dilution (FIG. 5E). At 50% dilution, signals approached the magnitude of the air-only tube signals with similarly noisy frequency spectra. This can be anticipated, as the extremes of microbubble concentrations are acoustically similar to a gas-only or water-only interface. As evident from FIGS. 5A-5F, the microbubble signal was contained within the same temporal span as the reflection from the tube (no portion of the total signal including the objective artifact extends beyond 75 μs), as it was a direct result of microbubbles interacting with the pulse travelling within the tube. As also demonstrated by FIG. 5, the bubble signals were highly correlated for each pressure—manifesting as a short 1 MHz pulse that begins near 66 μs with very similar amplitude across all pulses (data not shown).

FIGS. 5A-5F illustrate individual voltage traces received from microbubble samples of varying concentration and associated spectra. At each concentration, signal magnitude increased with incident pressure while retaining similar temporal characteristics. Signal magnitude increased from the lowest concentration tested of 0.05%, (FIGS. 5A and 5B) and maximized at 1% (FIGS. 5C and 5D). Past this, signal magnitude decreased up to the maximum concentration of 50% (FIGS. 5E and 5F).

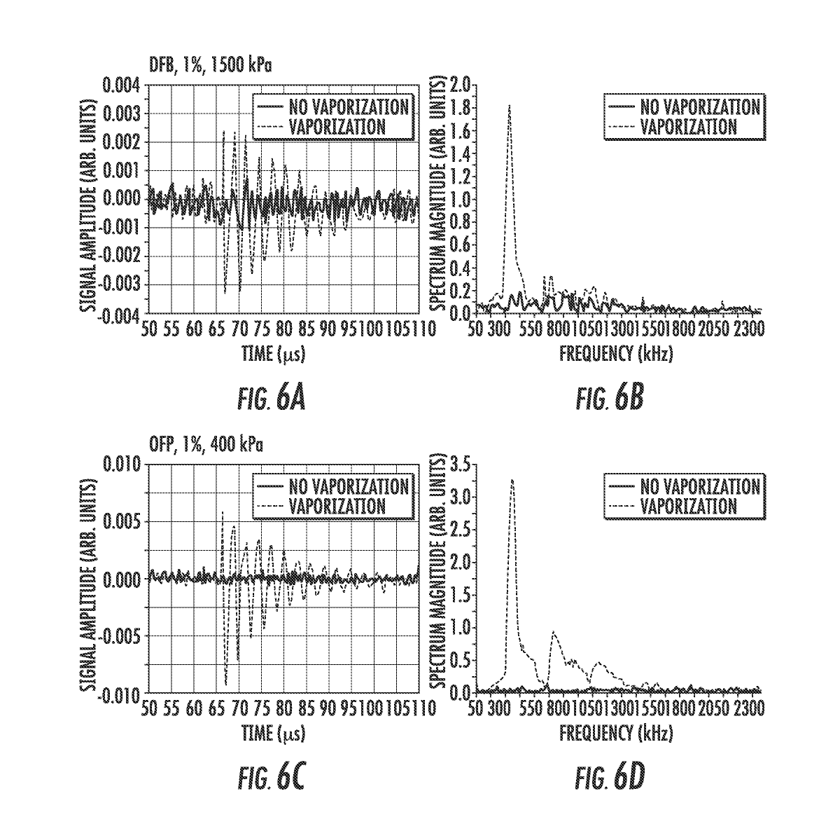

When volatile phase-change contrast agents were vaporized in the microcellulose tube, the difference in both the time and frequency domain compared to controls could easily be observed (FIGS. 6A-D). At low concentrations and/or low vaporization pressures, individual droplet vaporization traces could be isolated and compared to the signals received when no droplet vaporization occurred. Unlike the control cases, which manifested as short Gaussian-enveloped pulses between 64 μs and 75 μs with a wide frequency band centered around 1 MHz, droplet traces appeared as exponentially decaying sinusoids that often extended well beyond the 75 μs timepoint (FIG. 6A, 6C), similar to the decaying radial oscillation over 10-20 μs observed optically. As with the optical experiments capturing the initial rapid radial expansion of the vaporizing droplet, the maximum pressures recorded occurred at the very beginning of each droplet signal. Even when exposed to the same peak negative pressure, the voltage traces were highly variable from line to line with respect to both amplitude and frequency of the decaying sinusoid. In all cases, the pressure signal recorded began at the temporal location of the tube. In the frequency domain, sharp, narrowband peaks were present corresponding to the frequency of the decaying sinusoid (FIG. 6B, 6D). These narrowband peaks ranged from the low 100s of kHz to 1.3 MHz in this experimental setup—matching the experimental frequencies obtained in optical experiments (FIG. 3) well. At higher peak negative pressures and/or concentrations, the vaporization of multiple droplets produced voltage traces that were superimpositions of individual droplet pressure fields, and several individual narrowband peaks in the frequency domain could be observed corresponding to the individual droplets vaporized.

Although the frequency content of the droplets appeared generally independent of perfluorocarbon choice, the amplitude of the oscillations was significantly larger for OFP droplets than similarly sized DFB droplets (FIG. 6A, 6C), likely as a result of a more rapid initial expansion and greater overexpansion ratio upon vaporization. By the oscillation frequency experimental curve fit (FIG. 3), FIG. 6A shows the resonance (333 kHz) of a DFB bubble near 16.1 μm in diameter. By FIG. 2, this originated from a droplet near 3.1 μm in diameter. Similarly, the resonance (350 kHz) of the OFP bubble (FIG. 6C) originated from a bubble near 15.3 μm in diameter, or a droplet near 2.8 μm in diameter. Despite the similar sizes and the fact that a significantly higher pressure was used to vaporize the DFB droplet, the maximum amplitude of the OFP bubble oscillation was 2.8 times larger than the DFB bubble oscillation. Interestingly, the maximum amplitude observed in both cases occurred during the compression phase just after the initial expansion, and it was apparent that the steadily decaying sinusoids were exhibiting nonlinear oscillation behavior that generates harmonics in the frequency domain.

FIGS. 6A-6D illustrate signals produced by individual droplets vaporizing in the receive transducer's focus manifest as exponentially decaying sinusoids with very narrowband frequency content. Examples of DFB droplet vaporization (FIG. 6A) and the associated frequency spectrum (FIG. 6B). FIGS. 6C and 6D illustrate that OFP droplets oscillating at a similar frequency produce larger amplitude oscillations even when exposed to significantly lower incident pressure as a result of greater droplet volatility. In both FIGS. 6A and 6C, the blue trace shows the preceding pulse that did not produce a vaporized droplet for the same sample and pulse pressure.

Based on the optical results, larger droplets displace a larger volume of water during the radial oscillations at relatively similar speeds as smaller droplets, and should therefore generate larger amplitude pressure waves. In the acoustic detection scenario, this should result in lower frequency oscillation signals containing larger amplitudes of oscillation. However, this is not readily apparent from the raw data, as the frequency-dependent sensitivity of the receiver transducer must first be accounted for. The maximum sensitivity of the receiver transducer occurs for signals near 1.2 MHz. For signals near 330-350 kHz with the same amplitude, the received signal will be only 15% of the 1.2 MHz case. As a result, the raw signals shown in FIGS. 6A-6D had similar amplitude to the very largest bubble signals produced in any case once this factor is accounted for. By scaling the raw data in order to correct for the receiving transducer's frequency response, the relative amplitudes across the entire frequency range can be recovered.

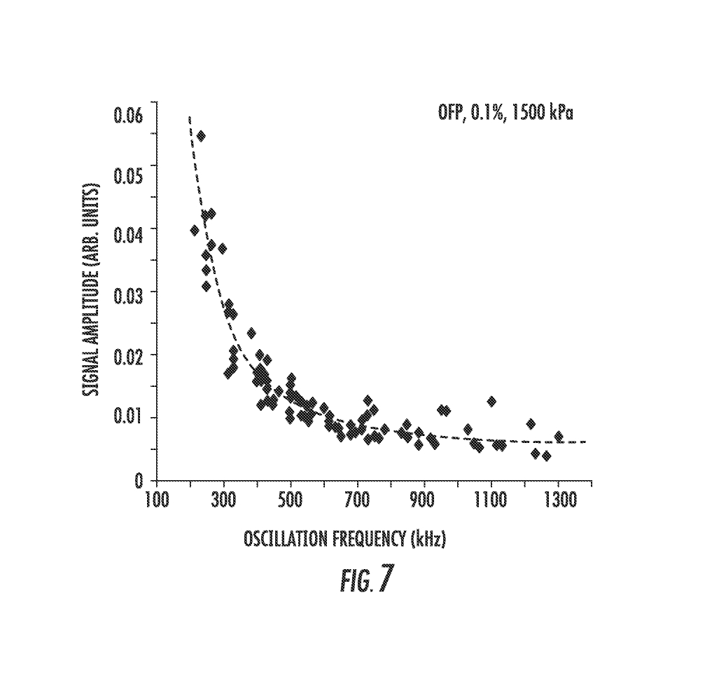

For example, the relationship between the amplitude of oscillation and the frequency of oscillation for single OFP droplets can be evaluated experimentally by exposing highly dilute samples of OFP droplets to peak negative pressures large enough to vaporize the majority of the droplets in the focal zone. Out of 200 vaporization pulses, 109 instances of the characteristic droplet oscillation were detected. Two of these were excluded, as they contained several spectral peaks, and did not represent single-droplet traces. For the remaining data set, mapping the amplitude of oscillation to the peak in the frequency domain revealed a power-law relationship of (f_{kHz})^{-2.072}, (r²=0.89, 95% confidence intervals of −2.403 and −1.741) (FIG. 7). As expected, the lower oscillation frequencies produced by bigger droplets result in significantly larger oscillation amplitudes. Here, frequency peaks were observed between 216 kHz and 1.3 MHz—corresponding to bubbles between 24.8 μm and 4.1 μm, or droplets between 4.5 μm and 0.7 μm in diameter (by the experimental relationship in FIG. 2).

FIG. 7 is a graph illustrating that, when scaled to correct for the receiver transducer's frequency-dependent sensitivity, the amplitude of single OFP droplet signals can be related to the frequency of oscillation by a power-law fit of (f_{kHz})^{-2.072}·(r²=0.89). A similar relationship can be experimentally derived for DFB droplets.

### 3.3. A Simple Spectral Approach to Droplet Detection

As the results above show, the general characteristics of signals produced by droplets when vaporized by a higher-frequency pulse can be easily distinguished from the control cases temporally and with regard to frequency content. In these tests, samples were collected for a single line of interrogation so that droplet signals could be localized axially. Extending this to a linear or matrix transducer array and collecting similar data on all channels simultaneously would allow for localization of the droplet vaporization signals in 2D or 3D space. This would have high utility for both diagnostic and therapeutic applications of PCCAs, such as localizing vaporization activity to correlate with in vivo PCCA-mediated drug delivery. For the purposes of developing the techniques demonstrated here into imaging strategies, many approaches are available. The temporal aspects of vaporized droplet signals would be distinct from the surrounding tissue and alternative ultrasound contrast agents, and could be isolated through correlation-based approaches such as spatial coherence (Lediju et al 2010) or matched-filtering (Misaridis and Jensen 2005). Alternatively, the signals generated from vaporizing droplets could be detected and distinguished from the surrounding tissue by only passing signals with narrowband frequency properties. These types of considerations will be the subject of future investigations.

In the remainder of this section, we implement a simple spectral approach to detect vaporization from droplets within a desired size range and demonstrate that the droplet vaporization signals can be distinguished from all control cases even at low concentrations and peak negative pressures. In this approach, the raw signals collected from each pulse ensemble are processed in three steps: wall-filtering, scaling to correct for receiving transducer frequency dependence, and frequency domain integration over a narrow frequency range related to the desired droplet sizes.

In the first processing step, the raw data ensemble obtained from the 200 pulses at each peak negative pressure is wall-filtered similar to conventional Doppler ultrasound processing in order to remove signals that are stationary from pulse-to-pulse. Here, the mean echo for each temporal location across all pulses is subtracted from that temporal location, leaving only signals that are transient from pulse to pulse. This largely eliminates the echoes returned from the water, air, and bubble samples, where there is very little variation from pulse to pulse. Next, the frequency spectrum of each wall-filtered time domain signal was obtained and the magnitude of the spectrum scaled to correct for the receive transducer's frequency-dependent sensitivity between 50 kHz and 2 MHz. Finally, the signals were bandpass filtered with a 100-order Butterworth FIR filter between 50 kHz and 700 kHz and integrated in this same frequency band to arrive at a final measure of magnitude for the band-limited signals. These edge frequencies correspond to bubbles between 7.7 μm and 107 μm in diameter, or droplets between 1.4 μm and 20.9 μm in diameter for both PFCs by the experimental fit in FIG. 2. It's worth noting here that the largest droplet characterized in the optical experiments was approximately 9.5 μm, and so there are likely to be very few droplets larger than this, if any, that contribute to the measure.

Applying this processing to the data as a function of concentration and peak negative pressure allows calculation of signal statistics for comparison between droplets and control cases. The largest value produced by any of the control cases after this processing was for the 50% bubble dilution at the highest peak negative pressure used. Droplet samples, on the other hand, produced a wide range of values as a result of the presence of lines with no droplet vaporization (values below the 50% bubble dilution reference) and those with droplet vaporization (values well above the 50% bubble dilution reference). Droplet signals can therefore be identified by choosing a control-based threshold. In this case, the 99^{th} percentile integrated spectral value obtained for the 50% bubble dilution case at the highest peak negative pressure was chosen as a reference, and any signals at least 10% greater than this were considered to be droplet signals. This threshold is highly conservative, as a 50% bubble concentration is not a physiologically relevant scenario for agents flowing in the bloodstream. However, for the purposes of this investigation it serves as a proof-of-concept that the droplet-based signals are distinct from any possible value of the control groups when processed in this manner.

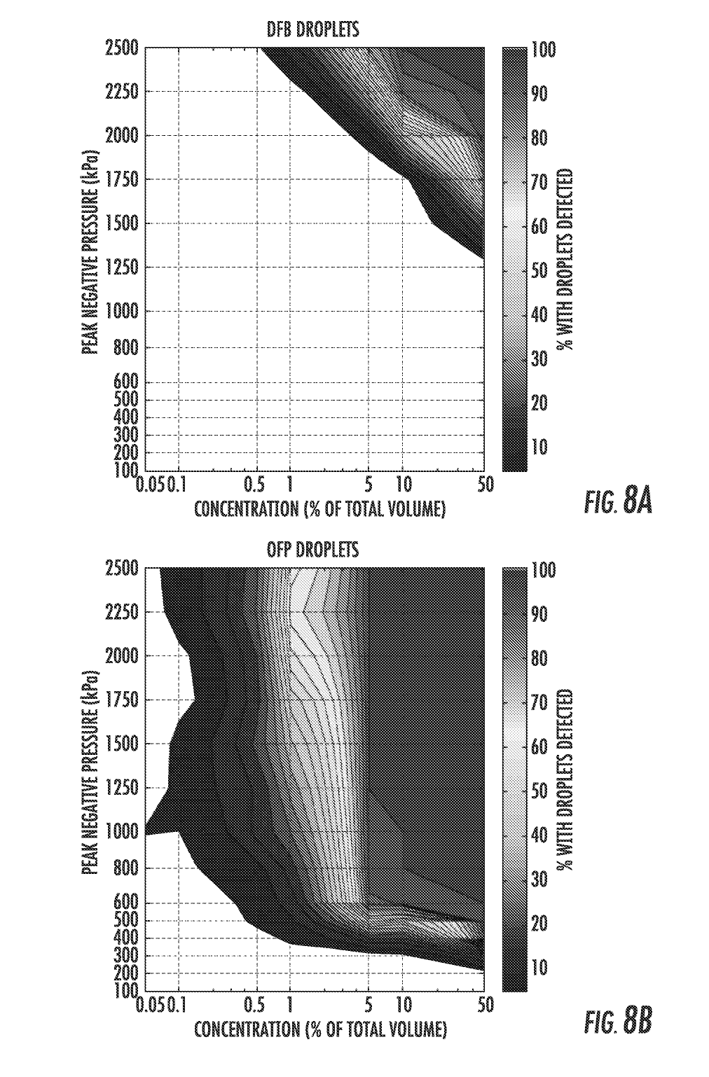

Applying this threshold to the 200 individual pressure traces captured at each concentration and peak negative pressure allows calculation of the likelihood of detecting droplets at the given flow rate for each combination of parameters (FIG. 8). Out of the 200 pulses for each test case, DFB droplets did not begin to show significant vaporization until the concentration was above 0.5% at the highest peak negative pressure. The number of pulses with droplet vaporization increased with concentration up to 10% concentration—after which every pulse exhibited droplet vaporization. At the higher concentrations, a significant number of DFB droplet signals were registered at lower peak negative pressures, illustrating the interplay of concentration and peak negative pressure in the stochastic process. It should be noted here that some individual droplet signals could be detected at lower concentrations and peak negative pressures than shown in FIG. 8 (such as the droplet trace in FIG. 6A), as this figure is limited to tests where 5% or more of the 200 lines exhibit droplet signals. However, for any combination of concentration and pressure where the peak negative pressure is 1250 kPa or less, very little droplet vaporization was captured. OFP samples, on the other hand, showed significant vaporization across a range of concentrations and peak negative pressures—reflecting the greatly reduced vaporization thresholds of OFP droplets compared to DFB. For nearly all combinations where concentration was above 0.5% and pressure was above 400 kPa, 5% or more of the samples showed droplet signals. No significant vaporization was detected below 200 kPa for any concentration, while at the lowest two concentrations used, peak negative pressures above 1000 kPa occasionally produced vaporization at the 5% level.

FIGS. 8A and 8B illustrate percentage of 200 sequential pulses where droplet signals are detected as a function of concentration and peak negative pressure of the incident 8 MHz ultrasound pulse. FIG. 8A illustrates that DFB droplets show little vaporization below the higher concentration/pressure combinations, while FIG. 6B illustrates that OFP droplets with much higher sensitivity to ultrasound show a significant amount of vaporization at low concentration/pressure combinations.

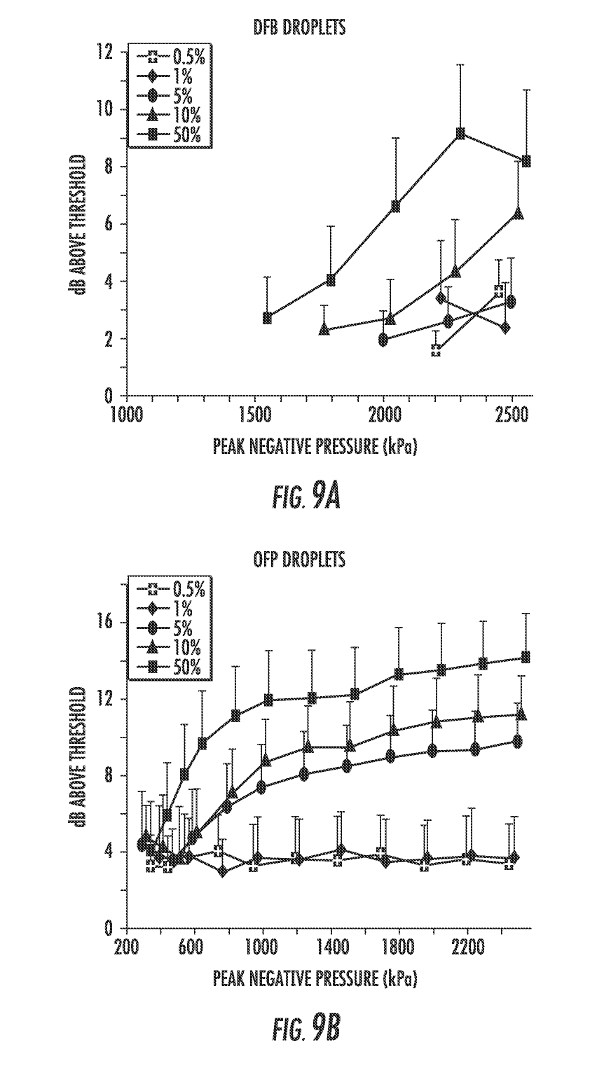

At concentration/pressure combinations where at least 3 droplet-specific signals were detected, statistics show that the spectral integration magnitude was dependent on PFC choice, as can be expected from earlier results. The DFB droplet signals at lower concentrations were on the order of 2 dB higher than the bubble threshold value, while higher concentrations produced signals upward of 9 dB over the bubble threshold value (FIG. 9A). The increase in signal strength observed for higher concentrations as a function of peak negative pressure is expected to be a result of a much larger number of droplets vaporizing in the focal zone (these are combinations that produce very high vaporization percentages in FIG. 8). For OFP droplets, concentrations 1% or below produced a consistent 4 dB increase over the bubble threshold regardless of the increase in vaporization pressure, while higher concentrations increased with vaporization pressure to values as high as 15 dB above the bubble threshold.

FIGS. 9A and 9B illustrate strength of detected droplet signals relative to 99^{th} percentile microbubble (50% dilution) signal magnitude after processing. FIG. 9A illustrates that DFB droplets exceed the threshold by 2 to 9 dB, depending on concentration and peak negative pressure. FIG. 9B illustrates that OFP droplets exceeded the threshold by between 4 and 15 dB, depending on concentration and peak negative pressure, highlighting the much larger signals produced by OFP droplets as a result of greater volatility. At concentrations 1% or below, signal strength was independent of pulse pressure as a result of fewer droplets vaporizing in the focal plane. At combinations of high concentration and high pulse pressure, the much greater number of vaporizing droplets resulted in an increase in signal strength. Note: The lowest concentrations (0.1% and 0.05%) omitted for clarity, and traces shifted slightly for clarity in error bars.

It should be noted here that FIGS. 9A and 9B show the strength of the droplet signals relative to the 99^{th} percentile bubble signal for the 50% dilution of bubbles. In vivo, a more relevant reference level may be the water-only microcellulose tube control, which mimics a blood-filled vessel. When compared to the 99^{th} percentile water-tube signal, DFB droplet signals were approximately 6 to 13 dB greater in magnitude, and OFP signals were approximately 8 to 19 dB greater in magnitude, depending on concentration.

### 3.4. Limitations and Future Directions

This study provides a preliminary demonstration that, under the right vaporization conditions, PCCAs emit size-dependent acoustic signals that can be detected. The subject matter described herein characterizes these phenomena both optically and acoustically and implements a specific droplet detection technique based on the results. However, there are some fundamental limitations to the study that warrant discussion.

The experimental relationships between bubble size and resonance frequency obtained by optical experiments is modeled well by the simple Minnaert resonance calculations (FIG. 3). However, this model neglects factors such as surface tension and shell properties that are likely to influence the physical phenomena captured here. In this study a classic damped harmonic oscillator model was implemented to account for some of these aspects, but the uncertainty in frequency resolution imposed by having only 24 frames maximum during optical experiments limits implementation of more robust models. Future refinement in the size/frequency relationship through alternative optical imaging equipment or simultaneous optical/acoustic capture may allow implementation of alternative resonance models in order to estimate physical properties of the droplets. Alternatively, the exponentially decaying sinusoidal pressure waveforms captured during acoustic interrogation may be used to estimate physical properties through the interaction of amplitude, frequency, and decay rate for droplets of varying size. The decay rate, in particular (as demonstrated in FIG. 6A-D), is directly related to thermal damping, which includes effects of Laplace pressure and the gas-dependent polytropic index (Ainslie and Leighton 2011). One aspect that warrants future study is the degree to which the initial vaporization pulse influences the vaporization phenomena observed. It is assumed in this study that the impact on overexpansion, oscillation, and final size of the 2-cycle 8 MHz pulse is not significant. Future studies with shorter, higher frequency pulses will reveal whether this is the case or not.

Frequency-banded detection techniques such as those used for FIG. 8 may be useful for determining thresholds of activation for different perfluorocarbon emulsions under various flow conditions and concentrations. Currently, there is no standardized method to evaluate practical in vivo activation parameters for PCCA formulations. Many studies measuring thresholds rely on acoustic techniques that are highly influenced by large outliers in the typically polydisperse size distributions, and alternative optical techniques of determining thresholds are limited with respect to sub-micron particles (Sheeran et al 2013). Using the techniques demonstrated here, the frequency bands corresponding to sizes of interest can be isolated in order to determine relative activation under given peak negative pressures. Therefore, these methods offer, for the first time, the potential to determine activation of droplets of a particular size when surrounded by droplets of different sizes through acoustic interrogation.

Here, the detection is shown only for a single point in space, and with a single transducer element. Ongoing efforts are focused on capturing these signals with imaging transducers (across many elements) in order to detect these signals and form images incorporating the spatial aspects of droplet vaporization. Ultimately, detection of these signals may enable real-time imaging of droplet activation that could be used for many diagnostic and therapeutic applications of medical ultrasound. Although the ability to distinguish these signals in a water bath does not directly indicate the ability to do so in tissue, several factors suggest detection of these signals in vivo would be likely. The frequencies of the natural resonance of droplets investigated here occur on the order of 100 kHz to 2.5 MHz—which is relatively low in the context of diagnostic medical ultrasound. Because the activation pulse is 8 MHz in frequency, tissue will scatter energy back towards the transducer at this same frequency as well as higher harmonics—but generally not at lower frequencies (Clarke and ter Haar 1999). Bubbles driven by the ultrasound pulse, on the other hand, will reflect back energy across a wide range of higher harmonics of 8 MHz, but generally not much below the ½ frequency harmonic (Forsberg et al 2000) other than broadband energy that occurs from microbubble destruction (Sassaroli and Hynynen 2007). As a result, neither microbubbles nor tissue will generate substantial acoustic waves on the order of 100 kHz to 3 MHz when pulsed at 8 MHz, and the signal from droplets of this size vaporizing in tissue will be detectable. Additionally, the signals returning from vaporized droplets will suffer less from frequency-dependent tissue attenuation than the higher frequency bubble and tissue signals (D'Astous and Foster 1986), and only have to travel one-way through the tissue to reach the transducer.

Although only the outliers in the distributions used here were investigated in order to pair optical and acoustic results (average droplet size in each suspension was sub-micron and not well-resolved optically), these techniques could be used to detect vaporization of droplets much smaller than those shown here. For instance, droplets near 200 nm in diameter would vaporize to form bubbles on the order of 1 μm in diameter. By the isothermal Minnaert approximation, these would generate acoustic signals with frequencies near 5-6 MHz. These frequencies would likely still be detectable through tissue, but they overlap with the bandwidth of the vaporization pulse transducer used here and therefore would be more difficult to discern from tissue and bubble signals. In order to maintain signal separation, a higher frequency vaporization pulse would be necessary, which may limit the ability to achieve droplet vaporization through a depth of tissue. In addition to separation between the vaporization pulse frequency and the oscillation frequencies, the underlying physics impose limits on the relationship between droplet oscillation frequency and the length of the vaporization pulse. In order to detect these unique signals, it is necessary that the activation pulse not extend into the oscillation period so that the resulting bubble is not ‘driven’ by the ultrasound pulse itself. The time to finish vaporizing depends directly on the droplet size, and so to detect very small droplets, very short pulses must be used to initiate vaporization. The activation pulse can be shortened by increasing the frequency, but frequencies much higher than 10-15 MHz do not provide sufficient penetration into tissue to be useful in human diagnostic imaging in deep tissues, and would only be relevant to superficial applications. Thus, with ultrasound as the activation source, there may be a practical limit to the smallest size of droplet detectable for medical ultrasound. Despite this, there are many therapeutic applications where droplets are large in order to deliver large payloads of drugs (Couture et al 2011) or enhance ablation (Zhang et al 2010, 2011).

One of the most interesting possibilities of these techniques is to activate the droplets with non-acoustic sources and detect the unique droplet signals with ultrasound. Other groups have recently demonstrated activation with lasers through primarily thermal mechanisms to enable applications in medical photoacoustics (Strohm et al 2011, Wilson et al 2012), and it has been suggested that droplet nucleation may be possible with ionizing radiation such as X-ray (Apfel 1998). In these cases, there may be little to no acoustic interference from the activation source, and so the droplet-specific signals may be received with high fidelity. Also, a non-acoustic source will not ‘drive’ the resulting bubbles with forced oscillations in the same manner as an acoustic source, and so oscillations from very small droplets on the order of 5 MHz or higher may also be isolated easily.

In the paragraphs above, we described the use of ‘phase-change’ contrast agents (or PCCAs) that begin as microscale or nanoscale liquid droplets and are ‘activated’ by ultrasound to transition into the gas state. This activation occurs as the ultrasound wave propagating through tissue causes a local pressure drop in the region of the PCCAs and causes the liquid inside the PCCAs to tip to the gas state. In this case, the type of compound typically used as the core of the PCCA is of the perfluorocarbon family (very stable, relatively high molecular weight compounds consisting of only carbon and fluorine). As the agents change state, they also expand as a result of the difference in density between the liquid and gas, resulting in a final diameter on the order of 5-6 times the original liquid particle diameter. This change in density, compressibility, and size is of interest to medical imaging researchers for a variety of diagnostic and therapeutic applications, many of which are progressing toward clinical use.

We also showed that because the agents produced are inherently volatile, they emit unique acoustic waves upon vaporization under certain conditions. Here “volatile” means that the PCCAs are composed of compounds that normally boil from the liquid state to the gas state at room temperature, but are stabilized in the liquid state during the processing so that they will remain in the liquid state at higher temperatures than usual. Because the agents are highly volatile, they expand rapidly after the transition is initiated, and the speed of expansion pushes the agents to overshoot past their final resting size and oscillate over the course of several microseconds until they reach the final resting size (see FIGS. 1C and 1D). This expansion and oscillation displace the surrounding water, which generates new acoustic waves that travel in all directions—including back toward the ultrasound transducer to be detected. These phenomena of overexpansion and oscillation have not been previously characterized experimentally anywhere in the literature, and so no existing patent has made claims on detecting activation of this type of agent through these mechanisms. Additionally, under certain activation conditions, the acoustic waves produced by the agents are completely independent of any acoustic signal generated by the ultrasound transducer, and so the signals can be isolated against typical acoustic return from tissue or alternative ultrasound contrast agents (detailed further below).

For example, when a very brief acoustic pulse is used to initiate vaporization, but does not last long enough to influence the oscillation period of the agent, the signals produced by the droplets can be acoustically detected independently of the activation pulse. These signals appear very different from signals that would be returned from tissue or standard ultrasound contrast agents with respect to both the time domain characteristics (FIGS. 6A and 6C) as well as the frequency domain (FIGS. 6B and 6D). As a result of the physical oscillation of the droplet, the received signals take the form of decaying sinusoids, and the amplitude and frequency of the oscillation depends directly on the size of the resulting bubble. In the frequency domain, the droplet signals form sharp peaks at the frequencies corresponding to the oscillation of the particle. In comparison, the signals returned from conventional ultrasound contrast agents appear as short sinusoidal pulses that have relatively broad frequency content (FIG. 5). These findings are especially interesting because it is possible to develop a direct link between the measured signals and the sizes of the particles that are being vaporized—which is currently a challenge in the field. In the Section 3.3: A Simple Spectral Approach to Droplet Detection, above, we detail one possible implementation of a droplet detection algorithm that can identify the presence of droplet vaporization, but we envision multiple approaches to this that would be useful for a variety of applications. Example detection schemes are detailed below.

### 4. TECHNIQUES FOR DETECTING SIGNAL(S) PRODUCED BY ULTRASOUND PHASE-CHANGE CONTRAST AGENTS

The following techniques assume that the signal produced by a phase transition event, a time-varying pressure waveform (110 in FIG. 1A, is converted to a time-varying voltage waveform by a device known as a transducer located some distance away from the location at which the phase transition event occurs. This transducer may be the same device that induced the phase transition event or a separate device. The signal produced by the phase transition event described in Section 3 may propagate many centimeters through a fluid such as water or solid media such as human or animal tissues in order to reach the transducer. The transducer itself may consist of a single device or an array of transducers producing multiple unique time-varying voltage signals at unique spatial locations. The transducer may be stationary or mechanically moved via a stepper motor or similar mechanism.

### 4.1 A Priori Detection Techniques

The signal is described as a sinusoid with an exponential decay. Knowledge of this signal shape can allow it to be compared with an expected signal or a “dictionary” of expected to signals to determine the most similar signal due to mathematical criteria such as correlation coefficient or other similarity metrics. This includes matched filtering techniques, in which a previously acquired or idealized version of the phase transition signal is used to detect the signal in the presence of noise (electronic noise, signals arising from conventional ultrasound contrast agents, or signals arising from tissue or other objects in the field of view producing acoustic signals). This also includes Wiener filtering, which minimizes the mean-squared error between the expected and acquired signals.

### 4.2 Other Time Domain Techniques

The phase transition signal has been observed to have unique temporal characteristics which enable it to be detected and distinguished from other signals. Specifically, the phase transition signal is temporally longer than the signal from a traditional contrast agent or from tissue but short-lived over multiple acquisitions or “frames.” That is, the described phase transition signal is long in “ultrasound fast time,” on the order of a few microseconds, but short in “ultrasound slow time,” on the order of a few frames. (Typical ultrasound imaging system frame rates are at least 30 frames per second but may be as high as thousands of frames per second.) Thus, the phase transition signal may be separated from other signals by subtracting stationary signals through the use of simple high-pass filters, often termed stationary echo cancelers or “wall” filters in ultrasound systems. The signal may also be separated from other signals by the use of speckle tracking or other algorithms which track motion over multiple acquisitions for the purpose of either enhancing or removing motion signals.

Other time domain techniques include those which analyze spatial or temporal similarity (via cross-correlation or similar mathematical metrics) may also be used to detect the phase transition signal, as it is produced by nanometer-sized droplet transforming into a micrometer-sized bubble and thus produces a spatially compact signal which is highly dissimilar from (i.e. uncorrelated with) signals produced by other spatially adjacent sources including tissue or microbubbles. That is, in the data typically acquired to form a medical ultrasound image or in the image itself, the phase transition signal might be detected by looking for signals which are highly dissimilar from all other signals. Conversely, signals produced by tissue in ultrasound images are known to be highly similar (or “coherent”) over short distances. Examples of these techniques include acoustic contrast factor, short-lag spatial coherence, and cross-correlation-based beamforming approaches.

### 4.3 Frequency Domain (Spectral) Techniques

As described in Section 3, the observed transition signal has frequency content which is unique from acoustic signals produced by either human/animal tissue or non-phase change contrast agents. Thus methods of isolating the signal produced by a phase transition event include separation from other signals via finite impulse response (FIR) or infinite impulse response (IIR) filtering or other filtering implemented by mathematical convolution with acquired signals in the time domain, multiplication with a frequency-domain representation of acquired signals or approximations thereof.

The phase transition signal may be isolated from other signals by analyzing signal power or amplitude in the frequency domain (as in Section 3) and determining the total or maximum power within one or more frequency bands.

Alternatively, the phase transition signal may be isolated from other signals on the basis of arrival time, propagation speed, or other techniques which rely on the phase (rather than the amplitude or power) of acquired signals. The phase transition signal may be detected and isolated from other signals on the basis of phase characteristics within one or more spectral bands relative to the phase at other spectral bands.

### 4.4 Combination of Frequency and Time Domain Techniques Including Time-Varying Frequency Techniques

Due to the described structure of the signal and the mechanism by which it is produced (a large initial expansion of the contrast agent followed by slowly-decaying oscillation due to damping by the contrast agent shell), frequency content of the acquired phase transition signal may vary over time. Thus the signal may be detected and isolated by analyzing the variation in frequency with respect to time. Conversely, frequency content of signals from both tissue and conventional contrast agent is expected to remain relatively constant over time. Techniques for performing this analysis include cepstral techniques and variations and approximations thereof. These techniques essentially look at the spectrum of a spectrum and separate an acquired signal based on the rate of change in various frequency bands.

### 4.5 Techniques Relying on Singular Value Decomposition, Eigenanalysis, and Mathematical Orthogonality

The phase transition signal may also be detected and isolated from signals arising from other sources by decomposing acquired signals into various bases by techniques such as eigenvalue decomposition or singular value decomposition. By determining a mathematical base or bases in which the phase transition signal is highly dissimilar or “orthogonal” to other signals, separation may be achieved. These techniques essentially map a signal into a domain other than the frequency domain (which decomposes on the basis of sinusoids).

### 4.6 Inverse Filtering

Spatiotemporal inverse filtering approaches rely on measuring or estimating a propagation operator in order to remove any effects imposed on the signal by the propagation medium (water, tissue, etc.). In theory, if the scattering function of the target and the frequency-dependent propagation operator are known or can be estimated, an image of the target can be reproduced with a high degree of accuracy by treating the wave propagation process as a complex filtering operation. Such an approach could be used to detect phase change contrast agent signals if they propagate through the medium in a different way from tissue or conventional contrast agent signals. Specifically, the observed low frequency and long temporal extent of the phase change contrast agent signal indicates a different scattering function than that of either tissue or convention contrast agents.

### 4.7 Two- and Three-Dimensional Imaging Techniques

All described techniques at detecting the phase transition signal can also be applied to provide two- or three-dimensional images or videos of phase transition events occurring, either alone or in combination with other ultrasound imaging modes such as B-mode, color/Power Doppler, M-mode, spectral Doppler, continuous wave Doppler, or other functional imaging techniques relying on ultrasound imaging data such as elastography techniques. Ultrasound images of the phase transition event may be formed by acquiring signals from individual transducers (mechanically scanned or an array), separating the phase transition signal using one of the described approaches, and combining signals from via conventional ultrasound beamforming (i.e. “delay-and-sum”), or adaptive beamforming approaches based on either measured or a priori information of the phase transition signal or its environment, or synthetic aperture approaches, or synthetic or parametric imaging approaches which form images based on mathematical values derived from acquired data. Alternatively, the phase transition signal may be detected and isolated after beamformation on fully or partially summed data, or after demodulation of summed data, or after envelope detection, or after compression of envelope-detected data.

### 4.8 Signals Arising Out of Other Mechanisms of Inducing Phase Transition

The described techniques for detecting signals produced phase transition techniques also apply to other, non-acoustic means of inducing phase transition in a phase change contrast agent, including imparting electromagnetic (i.e. optical) or x-ray energy to the phase change contrast agent, as well as any method that involves increasing local temperature until droplets vaporize.

### 5. CONCLUSION

In this study we demonstrate that, as a result of the phenomena of over-expansion and oscillation, unique acoustic signals are generated by vaporizing phase-change droplets that can be detected and isolated from other acoustic sources. Beyond physical studies, this technique may have wide applicability in real-time monitoring of therapeutic processes involving phase-change contrast agents in ultrasound as well as other modalities, and may result in new diagnostic techniques.

### Detection of Velocity of Fluid in which Phase Change Contrast Agents are Flowing

According to another aspect of the subject matter described herein, the velocity of a fluid in which a phase change contrast agent is flowing may be determined based on changes in the detected acoustic signatures over time. For example, any of the methods and systems described herein may be used to apply acoustic energy to a phase change contrast agent flowing in a fluid and detect the acoustic signature the phase change contrast agent after the phase change is induced. The acoustic signature may be measured at different time intervals, and changes in the acoustic signature over time may be measured. From the changes in the acoustic signature over time, velocity and/or direction of the fluid in which the phase change contrast agent is embedded may be determined.

In one example, the change in amplitude of the acoustic signature may be measured. If the amplitude decreases over time, the fluid may be determined to be flowing away from the point where the acoustic signatures are measured. If the amplitude increases over time, the fluid may be determined to be flowing towards the point where the acoustic signatures are measured. The rate of increase or decrease in amplitude may be used to determine the velocity of the fluid.

In another example, the change in phase of the acoustic signatures over time may be used to determine the velocity and/or direction of the fluid in which the phase change contrast agent is located.

In yet another example, the change in frequency of the signatures over time may be used to determine the velocity and/or direction of the fluid in which the phase change contrast agent is located. For example, using Doppler effect equations for frequency shifting of a moving sound source, it may be determined whether the fluid in which the phase change contrast agent is located is moving towards the measurement location, away from the measurement location, and the velocity at which the fluid is moving away from or towards the measurement location.

The disclosure of each of the following references is hereby incorporated herein by reference in its entirety.

### REFERENCES

Ainslie M A and Leighton T G 2011 Review of scattering and extinction cross-sections, damping factors, and resonance frequencies of a spherical gas bubble J. Acoust. Soc. Am. 130 3184-208
Apfel R E 1998 U.S. Pat. No. 5,840,276, Apfel Enterprises, Inc., Activatable infusable dispersions containing drops of a superheated liquid for methods of therapy and diagnosis
Asami R, Ikeda T, Azuma T, Umemura S and Kawabata K 2010 Acoustic Signal Characterization of Phase Change Nanodroplets in Tissue-Mimicking Phantom Gels Jpn. J. Appl. Phys. 49
Asami R and Kawabata K 2012 Repeatable vaporization of optically vaporizable perfluorocarbon droplets for photoacoustic contrast enhanced imaging Ultrason. Symp. (IUS), 2012 IEEE Int 1200-3
Bouakaz A, Frigstad S, Ten Cate F J and de Jong N 2002 Super harmonic imaging: a new imaging technique for improved contrast detection Ultrasound Med. Biol. 28 59-68
Calderon A J, Eshpuniyani B, Fowlkes J B and Bull J L 2010 A boundary element model of the transport of a semi-infinite bubble through a microvessel bifurcation Phys. Fluids 22 11
Choi J J and Coussios C-C 2012 Spatiotemporal evolution of cavitation dynamics exhibited by flowing microbubbles during ultrasound exposure. J. Acoust. Soc. Am. 132 3538-49
Choi J J, Pernot M, Small S a and Konofagou E E 2007 Noninvasive, transcranial and localized opening of the blood-brain barrier using focused ultrasound in mice. Ultrasound Med. Biol. 33 95-104
Clarke R L and ter Haar G R 1999 Production of harmonics in vitro by high-intensity focused ultrasound. Ultrasound Med. Biol. 25 1417-24
Coussios C C and Roy R A 2008 Applications of acoustics and cavitation to noninvasive therapy and drug delivery Annu. Rev. Fluid Mech. 40 395-420
Couture O, Faivre M, Pannacci N, Babataheri A, Servois V, Tabeling P and Tanter M 2011 Ultrasound internal tattooing Med. Phys. 38 1116-23
D'Astous F T and Foster F S 1986 Frequency dependence of ultrasound attenuation and backscatter in breast tissue. Ultrasound Med. Biol. 12 795-808
Dayton P A, Allen J S and Ferrara K W 2002 The magnitude of radiation force on ultrasound contrast agents J Acoust Soc Am 112 2183-92
Doinikov A A and Bouakaz A 2011 Review of shell models for contrast agent microbubbles. IEEE Trans. Ultrason. Ferroelectr. Freq. Control 58 981-93
Eshpuniyani B, Fowlkes J B and Bull J L 2008 A boundary element model of microbubble sticking and sliding in the microcirculation Int. J. Heat Mass Transf. 51 5700-11
Forsberg F, Shi W T and Goldberg B B 2000 Subharmonic imaging of contrast agents Ultrasonics 38 93-8
Gessner R and Dayton P A 2010 Advances in molecular imaging with ultrasound Mol Imaging 9 117-27
Gessner R, Lukacs M, Lee M, Cherin E, Foster F S and Dayton P A 2010 High-resolution, high-contrast ultrasound imaging using a prototype dual-frequency transducer: In vitro and in vivo studies IEEE Trans Ultrason Ferroelectr Freq Control 57 1772-81
Gramiak R and Shah P M 1968 Echocardiography of the aortic root Invest Radiol 3 356-66
Hurrell A 2004 Voltage to pressure conversion: are you getting ‘phased’ by the problem? J. Phys. Conf. Ser. 1 57
Ten Kate G L, Sijbrands E J, Valkema R, ten Cate F J, Feinstein S B, van der Steen A F, Daemen M J and Schinkel A F 2010 Molecular imaging of inflammation and intraplaque vasa vasorum: a step forward to identification of vulnerable plaques? J Nucl Cardiol 17 897-912
King D A, Malloy M J, Roberts A C, Haak A, Yoder C C and O'Brien W D 2010 Determination of postexcitation thresholds for single ultrasound contrast agent microbubbles using double passive cavitation detection. J. Acoust. Soc. Am. 127 3449-55
King D A and O'Brien W D 2011 Comparison between maximum radial expansion of ultrasound contrast agents and experimental postexcitation signal results. J. Acoust. Soc. Am. 129 114-21
Kripfgans O D, Fowlkes J B, Miller D L, Eldevik O P and Carson P L 2000 Acoustic droplet vaporization for therapeutic and diagnostic applications Ultrasound Med Biol 26 1177-89
Kruse D E and Ferrara K W 2005 A new imaging strategy using wideband transient response of ultrasound contrast agents IEEE Trans Ultrason Ferroelectr Freq Control 52 1320-9
Lediju M A, Trahey G E, Jakovlijevic M, Byram B C and Dahl J J 2010 Short-lag spatial coherence imaging 2010 IEEE Int. Ultrason. Symp. 987-90
Leighton T G 1994 The Acoustic Bubble (London: Academic Press)
Linstrom P J and Mallard W G 2013 NIST Chemistry WebBook, NIST Standard Reference Database Number 69
Martin K H and Dayton P A 2013 Current status and prospects for microbubbles in ultrasound theranostics Wiley Interdiscip. Rev. Nanomedicine Nanobiotechnology 5 329-45
Mattrey R F 1994 The Potential Role of Perfluorochemicals (PFCS) in Diagnostic Imaging Artif. Cells, Blood Substitutes Biotechnol. 22 295-313
Miller D L, Kripfgans O D, Fowlkes J B and Carson P L 2000 Cavitation nucleation agents for nonthermal ultrasound therapy J. Acoust. Soc. Am. 107 3480-6
Minnaert M 1933 On musical air-bubbles and the sounds of running water Philos. Mag. 16 235-48
Misaridis T and Jensen J A 2005 Use of modulated excitation signals in medical ultrasound. Part I: Basic concepts and expected benefits. IEEE Trans. Ultrason. Ferroelectr. Freq. Control 52 177-91
Needles A, Arditi M, Rognin N G, Mehi J, Coulthard T, Bilan-Tracey C, Gaud E, Frinking P, Hirson D and Foster F S 2010 Nonlinear contrast imaging with an array-based micro-ultrasound system Ultrasound Med. Biol. 36 2097-106
Pitt W G, Singh R N, Perez K X, Husseini G A and Jack D R 2013 Phase Transitions of Perfluorocarbon Nanoemulsion Induced with Ultrasound: A Mathematical Model Ultrason. Sonochem.
Qamar A, Wong Z Z, Fowlkes J B and Bull J L 2010 Dynamics of acoustic droplet vaporization in gas embolotherapy Appl Phys Lett 96 143702
Qamar A, Wong Z Z, Fowlkes J B and Bull J L 2012 Evolution of acoustically vaporized microdroplets in gas embolotherapy. J. Biomech. Eng. 134 031010
Rapoport N 2012 Phase-shift, stimuli-responsive perfluorocarbon nanodroplets for drug delivery to cancer Wiley Interdiscip. Rev. Nanomedicine Nanobiotechnology 4 492-510
Rapoport N, Nam K-H, Gupta R, Gao Z, Mohan P, Payne A, Todd N, Liu X, Kim T, Shea J, Scaife C, Parker D L, Jeong E-K and Kennedy A M 2011 Ultrasound-mediated tumor imaging and nanotherapy using drug loaded, block copolymer stabilized perfluorocarbon nanoemulsions J. Control. Release 153 4-15
Rapoport N Y, Kennedy A M, Shea J E, Scaife C L and Nam K H 2009 Controlled and targeted tumor chemotherapy by ultrasound-activated nanoemulsions/microbubbles J. Control. Release 138 268-76
Reznik N, Shpak O, Gelderblom E, Williams R, de Jong N, Versluis M and Burns P N 2013 The efficiency and stability of bubble formation by acoustic vaporization of submicron perfluorocarbon droplets Ultrasonics In Press
Reznik N, Williams R and Burns P N 2011 Investigation of Vaporized Submicron Perfluorocarbon Droplets as an Ultrasound Contrast Agent Ultrasound Med. Biol. 37 1271-9
Salgaonkar V a, Datta S, Holland C K and Mast T D 2009 Passive cavitation imaging with ultrasound arrays. J. Acoust. Soc. Am. 126 3071-83
Sassaroli E and Hynynen K 2007 Cavitation threshold of microbubbles in gel tunnels by focused ultrasound. Ultrasound Med. Biol. 33 1651-60
Sheeran P S and Dayton P A 2012 Phase-change contrast agents for imaging and therapy Curr. Pharm. Des. 18 2152-65
Sheeran P S, Luois S, Dayton P A and Matsunaga T O 2011 a Formulation and Acoustic Studies of a New Phase-Shift Agent for Diagnostic and Therapeutic Ultrasound Langmuir
Sheeran P S, Luois S H, Mullin L B, Matsunaga T O and Dayton P A 2012 Design of ultrasonically-activatable nanoparticles using low boiling point perfluorocarbons Biomaterials 33 3262-9
Sheeran P S, Matsunaga T O and Dayton P A 2013 Phase-transition thresholds and vaporization phenomena for ultrasound phase-change nanoemulsions assessed via high-speed optical microscopy Phys. Med. Biol. 58 4513
Sheeran P S, Wong V P, Luois S, McFarland R J, Ross W D, Feingold S, Matsunaga T O and Dayton P A 2011 b Decafluorobutane as a phase-change contrast agent for low-energy extravascular ultrasonic imaging Ultrasound Med Biol 37 1518-30
Shpak O, Kokhuis T J A, Luan Y, Lohse D, de Jong N, Fowlkes B, Fabiilli M and Versluis M 2013a Ultrafast dynamics of the acoustic vaporization of phase-change microdroplets J. Acoust. Soc. Am. 134 1610-21
Shpak O, Stricker L, Versluis M and Lohse D 2013b The role of gas in ultrasonically driven vapor bubble growth. Phys. Med. Biol. 58 2523-35
Streeter J E, Gessner R, Miles I and Dayton P A 2010 Improving sensitivity in ultrasound molecular imaging by tailoring contrast agent size distribution: in vivo studies Mol Imaging 9 87-95
Stride E P and Coussios C C 2010 Cavitation and contrast: the use of bubbles in ultrasound imaging and therapy Proc. Inst. Mech. Eng. Part H- Journal Eng. Med. 224 171-91
Strohm E, Rui M, Gorelikov I, Matsuura N and Kolios M 2011 Vaporization of perfluorocarbon droplets using optical irradiation Biomed. Opt. Express 2 1432-42
Whittingham T A 2005 Contrast-specific imaging techniques: technical perspective Contrast Media Ultrason. Basic Princ. Clin. Appl. 43-70
Wilson K, Homan K and Emelianov S 2012 Biomedical photoacoustics beyond thermal expansion using triggered nanodroplet vaporization for contrast-enhanced imaging Nat. Commun. 3 618
Wong Z Z, Kripfgans O D, Qamar A, Fowlkes J B and Bull J L 2011 Bubble evolution in acoustic droplet vaporization at physiological temperature via ultra-high speed imaging Soft Matter 7 4009-16
Ye T and Bull J L 2004 Direct Numerical Simulations of Micro-Bubble Expansion in Gas Embolotherapy J. Biomech. Eng. 126 745-59
Ye T and Bull J L 2006 Microbubble Expansion in a Flexible Tube J. Biomech. Eng. 128 554-63
Zhang M, Fabiilli M L, Haworth K J, Fowlkes J B, Kripfgans O D, Roberts W W, Ives K A and Carson P L 2010 Initial Investigation of Acoustic Droplet Vaporization for Occlusion in Canine Kidney Ultrasound Med. Biol. 36 1691-703
Zhang M, Fabiilli M L, Haworth K J, Padilla F, Swanson S D, Kripfgans O D, Carson P L and Fowlkes J B 2011 Acoustic Droplet Vaporization for Enhancement of Thermal Ablation by High Intensity Focused Ultrasound Acad Radiol
Zhang P and Porter T 2010 An in vitro study of a phase-shift nanoemulsion: a potential nucleation agent for bubble-enhanced HIFU tumor ablation Ultrasound Med Biol 36 1856-66

It will be understood that various details of the subject matter described herein may be changed without departing from the scope of the subject matter described herein. Furthermore, the foregoing description is for the purpose of illustration only, and not for the purpose of limitation.

What is claimed is:

1. A method for acoustic detection of activated phase-change contrast agents, the method comprising:
initiating a phase transition in a phase change contrast agent causing a change of the diameter of the activated phase change contrast agent; and
detecting an acoustic signature of the change of diameter of the activated phase change contrast agent.

2. The method of claim 1 wherein the phase change contrast agent comprises an encapsulated liquid and wherein initiating the phase transition comprises causing at least some of the liquid to change to a gaseous state.

3. The method of claim 2 wherein the liquid comprises a fluorocarbon, a hydrocarbon, a hydrofluorocarbon, or a hydrohalocarbon.

4. The method of claim 1 wherein initiating a phase transition in the phase change contrast agent comprises providing at least one of: heat energy, light energy, and mechanical energy to the phase change contrast agent.

5. The method of claim 1 wherein initiating a phase transition in the phase change contrast agent comprises using ultrasonic energy having a frequency selected from a range of 20 kHz to 100 MHz.

6. The method of claim 1 wherein detecting the acoustic signature comprises detecting an oscillation in the diameter of the activated phase change contrast agent.

7. The method of claim 5 wherein the detected oscillation is a damped sinusoidal oscillation having characteristic parameters.

8. The method of claim 5 comprising detecting the size of the activated phase change contrast agent based on at least one of:
a frequency of the detected oscillation;
an amplitude of the detected oscillation; and
a phase of the detected oscillation.

9. The method of claim 1 comprising detecting a location of the activated phase change contrast agent based on the detected acoustic signature.

10. The method of claim 1 comprising determining a number of activated phase change contrast agents based on a characteristic of the detected signatures.

11. The method of claim 1 wherein detecting the acoustic signature comprises detecting the acoustic signature using an ultrasonic transducer having a frequency range from 20 kHz to 100 MHz.

12. A system for acoustic detection of activated phase-change contrast agents and the activation of same, the system comprising:
an energy source for providing activation energy that activates a phase change contrast agent, causing a change of the diameter of the activated phase change contrast agent;
a detector for detecting an acoustic signature of the change of diameter of the activated phase change contrast agent.

13. The system of claim 12 wherein the phase change contrast agent comprise a shell containing a liquid and wherein initiating the phase transition comprises causing at least some of the liquid to change to a gaseous state.

14. The system of claim 13 wherein the liquid comprises a fluorocarbon, a hydrocarbon, a hydrofluorocarbon, or a hydrohalocarbon.

15. The system of claim 12 wherein the energy source provides at least one of heat energy, light energy, and mechanical energy to the phase change contrast agent.

16. The system of claim 12 wherein the energy source provides ultrasonic energy having a frequency selected from a range of 20 kHz to 100 MHz.

17. The system of claim 12 wherein detecting the acoustic signature comprises detecting an oscillation in the diameter of the activated phase change contrast agent and wherein the system further comprises a controller for determining a characteristic of the phase change contrast agent or a fluid in which the phase change contrast agent is located based on the acoustic signature.

18. The system of claim 17 wherein the detected oscillation is a damped sinusoidal oscillation having characteristic parameters.

19. The system of claim 17 wherein the controller determines the size of the activated phase change contrast agent based on at least one of:
a frequency of the detected oscillation;
an amplitude of the detected oscillation; and
a phase of the detected oscillation.

20. The system of claim 17 wherein the controller determines a location of the activated phase change contrast agent based on the detected acoustic signature.

21. The system of claim 17 wherein the controller determines a number of activated phase change contrast agents based on a characteristic of the detected signature.

22. The system of claim 17 wherein the detector comprises an ultrasonic transducer having a frequency range from 20 kHz to 100 MHz.

23. A non-transitory computer readable medium having stored thereon executable instructions that when executed by the processor of a computer control the computer to perform steps comprising:
initiating a phase transition in a phase change contrast agent causing a change of the diameter of the activated phase change contrast agent; and
detecting an acoustic signature of the change of diameter of the activated phase change contrast agent.

24. A device for acoustic detection of activated phase-change contrast agents, the device comprising:
a first transducer comprising at least one element for producing ultrasound energy at a first frequency to initiate a phase transition of a phase change contrast agent causing a change of the diameter of the activated phase change contrast agent; and
a second transducer comprising at least one element for detecting an acoustic signature of the change of diameter of the activated phase change contrast agent.

25. The device of claim 24, where the first and second transducers have −6 dB bandwidths which do not overlap

26. The device of claim 24, where the first and second transducers have −12 dB bandwidths which do not overlap.

27. A method for acoustic detection of activated phase change contrast agents, the method comprising:
initiating a phase transition in a phase change contrast agent causing the phase change transfer agent to transition from a droplet in a liquid phase to a bubble in a gaseous phase and causing the bubble to over-expand and then oscillate in size with decaying amplitude; and
detecting an acoustic signature of the activated phase change contrast agent based on the overexpansion and decayed oscillation amplitude.

28. A method for measuring at least one of velocity and direction of a fluid in which an activated phase-change contrast agent is flowing, the method comprising:
initiating a phase transition in a phase change contrast agent causing a change of the diameter of the activated phase change contrast agent;
detecting an acoustic signature of the change of diameter of the activated phase change contrast agent; and
detecting a change in the acoustic signature of the activated phase change contrast agent over successive time samples;
determining at least one of a velocity and a direction of a fluid in which the activated phase-change contrast agent is flowing based on the change in the acoustic signature over the successive time samples.

29. The method of claim 28 where the change in acoustic signature comprises a change in amplitude of the acoustic signature over time.

30. The method of claim 28 wherein the change in acoustic signature comprises a change in phase of the acoustic signature over time.

31. The method of claim 28 wherein the change in acoustic signature comprises a change in frequency of the acoustic signature over time.

32. The method of claim 28 wherein the phase change contrast agent comprises an encapsulated liquid and wherein initiating the phase transition comprises causing at least some of the liquid to change to a gaseous state.

33. The method of claim 32 wherein the liquid comprises a fluorocarbon, a hydrocarbon, a hydrofluorocarbon, or a hydrohalocarbon.

34. The method of claim 28 wherein initiating a phase transition in the phase change contrast agent comprises providing at least one of: heat energy, light energy, and mechanical energy to the phase change contrast agent.

35. The method of claim 28 wherein initiating a phase transition in the phase change contrast agent comprises using ultrasonic energy having a frequency selected from a range of 20 kHz to 100 MHz.

36. The method of claim 28 wherein detecting the acoustic signature comprises detecting an oscillation in the diameter of the activated phase change contrast agent.

37. The method of claim 36 wherein the detected oscillation is a damped sinusoidal oscillation having characteristic parameters.

38. The method of claim 36 comprising detecting the size of the activated phase change contrast agent based on at least one of:
a frequency of the detected oscillation;
an amplitude of the detected oscillation; and
a phase of the detected oscillation.

39. The method of claim 28 comprising detecting a location of the activated phase change contrast agent based on the detected acoustic signature.

40. The method of claim 28 comprising determining a number of activated phase change contrast agents based on a characteristic of the detected signatures.

41. The method of claim 28 wherein detecting the acoustic signature comprises detecting the acoustic signature using an ultrasonic transducer having a frequency range from 20 kHz to 100 MHz.

42. A system for measuring at least one of velocity and direction of a fluid in which an activated phase-change contrast agent is flowing, the system comprising:
an energy source for initiating a phase transition in a phase change contrast agent causing a change of the diameter of the activated phase change contrast agent;
a detector for detecting an acoustic signature of the change of diameter of the activated phase change contrast agent; and
a controller for determining a change in the acoustic signature of the activated phase change contrast agent over successive time samples and for determining at least one of a velocity and a direction of a fluid in which the activated phase-change contrast agent is flowing based on the change in the acoustic signature over the successive time samples.

43. The system of claim 42 wherein the change in acoustic signature comprises a change in amplitude of the acoustic signature over time.

44. The system of claim 42 wherein the change in acoustic signature comprises a change in phase of the acoustic signature over time.

45. The system of claim 42, wherein the change in acoustic signature comprises a change in frequency of the acoustic signature over time.

46. The system of claim 42 wherein the phase change contrast agent comprises an encapsulated liquid and wherein initiating the phase transition comprises causing at least some of the liquid to change to a gaseous state.

47. The system of claim 46 wherein the liquid comprises a fluorocarbon, a hydrocarbon, a hydrofluorocarbon, or a hydrohalocarbon.

48. The system of claim 42 wherein initiating a phase transition in the phase change contrast agent comprises providing at least one of: heat energy, light energy, and mechanical energy to the phase change contrast agent.

49. The system of claim 42 wherein initiating a phase transition in the phase change contrast agent comprises using ultrasonic energy having a frequency selected from a range of 20 kHz to 100 MHz.

50. The system of claim 42 wherein detecting the acoustic signature comprises detecting an oscillation in the diameter of the activated phase change contrast agent.

51. The system of claim 50 wherein the detected oscillation is a damped sinusoidal oscillation having characteristic parameters.

52. The system of claim 51 wherein the controller is configured to determine the size of the activated phase change contrast agent based on at least one of:
a frequency of the detected oscillation;
an amplitude of the detected oscillation; and
a phase of the detected oscillation.

53. The system of claim 42 wherein the controller is configured to determine a location of the activated phase change contrast agent based on the detected acoustic signature.

54. The system of claim 42 wherein the controller is configured to determine a number of activated phase change contrast agents based on a characteristic of the detected signatures.

55. The system of claim 42 wherein detecting the acoustic signature comprises detecting the acoustic signature using an ultrasonic transducer having a frequency range from 20 kHz to 100 MHz.
